(19) United States
(12) Patent Application Publication
(10) Pub. No.: US 2026/0283972 A1
(43) Pub. Date: Sep. 24, 2026

(54) A COMPOUND FOR PREPARING LIPID NANOPARTICLES ENCAPSULATING AN AGENT, NANOPARTICLE COMPOSITION COMPRISING SAID COMPOUND AND RELATED METHODS THEREOF

(71) Applicant: AGENCY FOR SCIENCE, TECHNOLOGY AND RESEARCH, Singapore (SG)

(72) Inventors: Yi Yan YANG, Singapore (SG); Jin Yan Melgious ANG, Singapore (SG); QiangQiang WANG, Singapore (SG); Zhengjie WU, Singapore (SG)

(73) Assignee: AGENCY FOR SCIENCE, TECHNOLOGY AND RESEARCH, Singapore (SG)

(21) Appl. No.: 19/469,989

(22) PCT Filed: Mar. 27, 2024
(86) PCT No.: PCT/SG2024/050182

§ 371 Date: Sep. 26, 2025

(30) Foreign Application Priority Data
Mar. 27, 2023 (SG) 10202300816Y

(51) Int. Cl. A61K 9/51; A61K 38/12; A61P 31/04; C07K 1/107; C07K 7/06

(52) U.S. Cl. CPC A61K 9/5123; A61K 9/5146; A61K 9/5169; A61K 38/12; A61P 31/04; C07K 1/1077; C07K 7/06

(57) ABSTRACT

There is provided a compound comprising a structure represented by general formula (1) or ionized forms thereof for preparing lipid nanoparticles encapsulating a cationic therapeutic and/or cationic prophylactic agent: wherein R1 and R2 are each independently a hydrophobic group; X comprises an optionally substituted linear aliphatic, optionally substituted branched aliphatic and/or optionally substituted cyclic hydrocarbons; and A comprises a peptide sequence of 1-5 anionic amino acids.

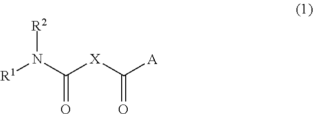

### TECHNICAL FIELD

The present disclosure relates broadly to a compound for preparing lipid nanoparticles encapsulating an agent and a method of preparing said compound. The present disclosure also relates to a nanoparticle composition comprising said compound and related methods and uses.

### BACKGROUND

Increasing resistance to therapeutic and/or prophylactic agents (e.g., commonly used drugs such as antibiotics) is of significant concern to global health as currently available therapeutic and/or prophylactic agents are becoming inefficacious against resistant infectious pathogens. One of the greatest concerns is the continuing escalation of resistance to Gram-negative bacteria resulting in the endemic presence of multidrug-resistant (MDR) and extremely drug-resistant (XDR) pathogens. Infections caused by MDR/XDR Gram-negative bacteria include pneumonia and respiratory infections which are associated with high rates of mortality. With the rapid rise in appearance of Gram-negative multidrug-resistant (MDR) and extremely drug-resistant (XDR) infections, there is renewed interest in using some of the earlier antibiotics that are known to be efficacious with excellent antibacterial activity such as polymyxin B. However, many of such therapeutic and/or prophylactic agents are toxic and can cause acute nephrotoxicity and neurotoxicity upon systemic administration. The toxicity problem is further compounded by their inherent modest stability in the bloodstream, thereby necessitating higher drug doses to be administered in patients for treatment.

Consequently, new strategies are required in the loading and delivery of therapeutic and/or prophylactic agents (e.g., drugs such as antibiotics). Recently, lipid-based nanocarriers have been recognized as one of the most promising delivery systems to encapsulate many therapeutic cargos. Empowered by the advent of microfluidics technology, on-chip preparation of lipid-based nanocarriers has shown their great potential to control manufacturing in the laboratory and industrial settings, and reproduce the desired physical properties of the nanoparticles like size, polydispersity, morphology, and lamellarity with ease. However, currently available delivery systems have several disadvantages and drawbacks, and are far from desirable. Particularly, there have been reports of adverse health effects and cytotoxicity associated with the use of lipid nanoparticles for delivery. Accordingly, a safe, stable and efficacious delivery system remains a challenge.

In view of the above, there is a need to address or at least ameliorate the above-mentioned problems. In particular, there is a need to provide a compound and/or nanoparticle composition for a substantially safe and stable, and/or efficacious delivery of therapeutic and/or prophylactic agents.

### SUMMARY

In one aspect, there is provided a compound comprising a structure represented by general formula (1) or ionized forms thereof for preparing lipid nanoparticles encapsulating a cationic therapeutic and/or cationic prophylactic agent:

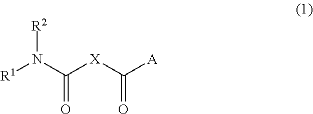

wherein
R¹ and R² are each independently a hydrophobic group;
X comprises an optionally substituted linear aliphatic, optionally substituted branched aliphatic and/or optionally substituted cyclic hydrocarbons; and
A comprises a peptide sequence of 1-5 anionic amino acids.

In one embodiment, the structure is represented by general formula (1A):

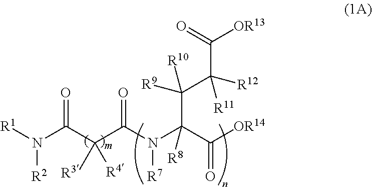

wherein
R^{3′}, R^{4′}, R⁷ to R¹⁴ are each independently selected from the group consisting of H, optionally substituted alkyl, optionally substituted alkenyl, optionally substituted alkynyl, and combinations thereof;
m is 1, 2, 3, 4 or 5; and
n is 1, 2, 3, 4 or 5.

In one embodiment, the hydrophobic group at R¹ and R² each independently comprises optionally substituted alkyl.

In one embodiment, the compound is an ionized form of general formula (1).

In one embodiment, the compound is an ionized form of general formula (1A), where —C(═O)OR¹³ and/or —C(═O)OR¹⁴ has been ionized to become a negatively charged group.

In one embodiment, the cationic therapeutic and/or cationic prophylactic agent comprises an antimicrobial agent.

In one embodiment, the cationic therapeutic and/or cationic prophylactic agent comprises polymyxin.

In another aspect, there is provided a method of preparing a compound represented by general formula (1) as disclosed herein, the method comprising:
(a-i) reacting a resin with a fully protected (i.e., N- and C-protected) amino acid to attach the fully protected amino acid to the resin;
(a-ii) removing N-protecting group from the fully protected amino acid to obtain a partially protected (i.e., C-protected) amino acid;
(a-iii) reacting the partially protected (i.e., C-protected) amino acid with another fully protected amino acid to attach the fully protected amino acid to the partially protected amino acid via the N-terminus to obtain a resin-supported fully protected peptide represented by general formula (2):

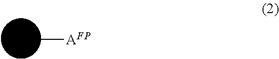

wherein
● represents resin; and
A^{FP} represents fully protected peptide (e.g., oligopeptide);
(a-iv) removing N-protecting group from the resin-supported fully protected peptide represented by general formula (2) to obtain a resin-supported partially protected (i.e. C-protected) peptide represented by general formula (3):

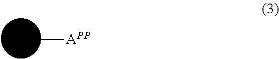

wherein
● represents resin; and
A^{PP} represents partially protected peptide (e.g., oligopeptide);
(a-v) reacting the resin-supported partially protected (i.e., C-protected) peptide represented by general formula (3) with an acid anhydride represented by general formula (4) to obtain a first intermediate compound represented by general formula (5);

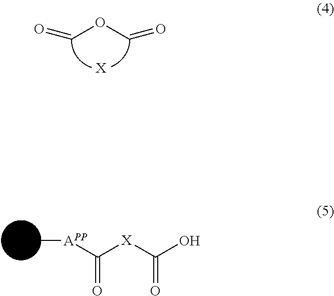

(a-vi) reacting the first intermediate compound represented by general formula (5) with an amine compound represented by general formula (6) to obtain a second intermediate represented by general formula (7); and

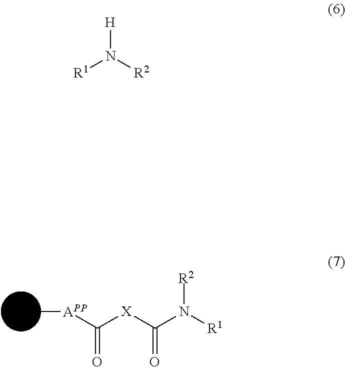

(a-vii) removing the resin from the second intermediate represented by general formula (7) to obtain a compound represented by general formula (1); and

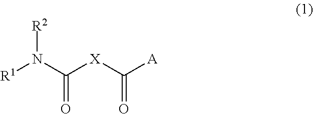

(a-viii) optionally ionizing A to become a negatively charged group.

In one embodiment, the method further comprises repeating step (a-ii) and/or step (a-iii) until an oligopeptide of a desired length is obtained.

In another aspect, there is provided a nanoparticle composition for delivery of a cationic therapeutic and/or cationic prophylactic agent, the nanoparticle composition comprising:
a compound as disclosed herein; and
a cationic therapeutic and/or cationic prophylactic agent that is encapsulated in said compound as disclosed herein.

In one embodiment, the charge ratio of the cationic therapeutic and/or cationic prophylactic agent that is encapsulated in the compound as disclosed herein to said compound is from 1:1 to 10:1.

In one embodiment, the composition further comprises:
(a) helper lipid;
(b) sterol; and
(c) polyethylene glycol (PEG)-modified lipid.

In one embodiment, the cationic therapeutic and/or cationic prophylactic agent, helper lipid, sterol, compound represented by general formula (1), and PEG-modified lipid are mixed at a molar ratio of 10-120:3-30:10-130:2-30:0.5-10.

In one embodiment, the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin and combinations thereof.

In one embodiment, the sterol is selected from cholesterol, fecosterol, sitosterol, ergosterol, campesterol, stigmasterol, brassicasterol, avenasterol and combinations thereof.

In one embodiment, the PEG-modified lipid is selected from PEG-modified phosphatidylethanolamines, PEG-modified phosphatidic acids, PEG-modified ceramides, PEG-modified dialkylamines, PEG-modified diacylglycerols, PEG-modified dialkylglycerols, or the like or combinations thereof. Examples of PEG-modified/PEGylated lipid include, but is not limited to, 2-[(polyethylene glycol)-2000]-N,N-ditetradecylacetamide (ALC-0159), R-3-[(w-methoxy-poly(ethylene glycol) 2000) carbamoyl]-1,2-dimyristyloxlpropyl-3-amine (PEG-c-DOMG), 3-N-[(ω-methoxypoly(ethyleneglycol) 2000) carbamoyl]-1,2-dimyristyloxy-propylamine (PEG-S-DMG), PEG-DMPE (1,2-dimyristoyl-sn-glycero-3-phosphoethanolamine-N-[(polyethylene glycol)-methoxy](sodium salt)), PEG-DPPC, PEG-DSPE lipid and combinations thereof.

In one embodiment, the nanoparticle composition comprises nanoparticles having an average particle size of from 20 nm to 200 nm.

In one embodiment, the nanoparticle composition comprises nanoparticles having a zeta potential of from −20 mV to +20 mV.

In one embodiment, the nanoparticle composition comprises nanoparticles having a minimum bactericidal concentration (MBC) to minimum inhibitory concentration (MIC) ratio that is no more than 6.

In another aspect, there is provided the nanoparticle composition as disclosed herein for use in medicine.

In another aspect, there is provided the nanoparticle composition as disclosed herein for use in the treatment or prophylaxis of a disease, disorder or condition in a subject in need thereof.

In another aspect, there is provided use of a nanoparticle composition as disclosed herein in the manufacture of a medicament for treatment or prophylaxis of a disease, disorder or condition in a subject in need thereof.

In another aspect, there is provided a method of treating or preventing a disease, disorder or condition in a subject in need thereof, the method comprising administering a therapeutically effective amount of the nanoparticle composition as disclosed herein to the subject.

In another aspect, there is provided a method of treating or preventing a bacterial infection in a subject in need thereof, the method comprising administering a therapeutically effective amount of the nanoparticle composition as disclosed herein to the subject.

In one embodiment, the disease, disorder or condition is mediated by Gram-negative bacteria.

In one embodiment, the Gram-negative bacteria is selected from the group consisting of Escherichia coli, Pseudomonas aeruginosa, and combinations thereof.

### Definitions

The term “particle” as used herein broadly refers to a discrete entity or a discrete body. The particle described herein can include an organic, an inorganic, a composite particle or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, the term “size” when used in the context of nanoparticle can refer to the diameter of the nanoparticle although it is not limited as such. In various embodiments, when the particle is substantially spherical, the term “size” can refer to the diameter of the particle; or when the particle is substantially non-spherical, the term “size” can refer to the largest length of the particle.

The term “nano” as used herein is to be interpreted broadly to include dimensions in a nanoscale, i.e., less than about 1000 nm, about 1 nm to less than about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, or from about 1 nm to about 100 nm. Accordingly, the term “nanostructures”, “nanoparticles”, “nanomaterials” and the like as used herein may include structures that have at least one dimension in the range of no more than said range. The term “nanostructures”, “nanoparticles”, “nanomaterials” and the like as used herein may include structures that have at least one dimension that is no more than about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm.

The term “micro” as used herein is to be interpreted broadly to include dimensions from about 1 micron to about 1000 microns, about 1 micron to less than about 1000 microns, about 1 micron to about 900 microns, about 1 micron to about 800 microns, about 1 micron to about 700 microns, about 1 micron to about 600 microns, about 1 micron to about 500 microns, about 1 micron to about 400 microns, about 1 micron to about 300 microns, about 1 micron to about 200 microns, or from about 1 micron to about 100 microns.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases, symptoms and disorders. A medical condition also includes a body's response to a disease or disorder, e.g., inflammation. Those in need of such treatment include those already with a medical condition as well as those prone to getting the medical condition or those in whom a medical condition is to be prevented.

As used herein, the term “therapeutically effective amount” of a compound is intended to refer to an amount that is sufficient or capable of preventing or at least slowing down (lessening) a medical condition, such as infectious/contagious diseases, viral infections (i.e. diseases caused by virus), bacterial infections (i.e. diseases caused by bacteria), fungal infections (i.e. diseases caused by fungi), parasitic infections (i.e. diseases caused by parasite), respiratory diseases, or the like or combinations thereof. The disease, disorder or condition may be mediated by drug resistant bacteria and/or multidrug-resistant bacteria such as Gram-negative bacteria selected from Acinetobacter baumannii, Acinetobacter haemolyticus, Actinobacillus actinomycetemcomitans, Aeromonas hydrophila, Bacteroides fragilis, Bacteroides the ataioatamicron, Bacteroides distasonis, Bacteroides ovatus, Bacteroides vulgatus, Bordetella pertussis, Brucella melitensis, Burkholderia cepacia, Burkholderia pseudomallei, Burkholderia mallei, Prevotella corporis, Prevotella intermedia, Prevotella endodontalis, Porphyromonas asaccharolytica, Campylobacter jejuni, Campylobacter coli, Campylobacter fetus, Citrobacter freundii, Citrobacter koseri, Edwarsiella tarda, Eikenella corrodens, Enterobacter cloacae, Enterobacter aerogenes, Enterobacter agglomerans, Escherichia coli, Francisella tularensis, Haemophilus influenzae, Haemophilus ducreyi, Helicobacter pylori, Kingella kingae, Klebsiella pneumoniae, Klebsiella oxytoca, Klebsiella rhinoscleromatis, Klebsiella ozaenae, Legionella penumophila, Moraxella catarrhalis, Morganella morganii, Neisseria gonorrhoeae, Neisseria meningitidis, Pasteurella multocida, Plesiomonas shigelloides, Proteus mirabilis, Proteus vulgaris, Proteus penneri, Proteus myxofaciens, Providencia stuartii, Providencia rettgeri, Providencia alcalifaciens, Pseudomonas aeruginosa, Pseudomonas fluorescens, Salmonella typhi, Salmonella paratyphi, Serratia marcescens, Shigella flexneri, Shigella boydii, Shigella sonnei, Shigella dysenteriae, Stenotrophomonas maltophilia, Streptobacillus moniliformis, Vibrio cholerae, Vibrio parahaemolyticus, Vibrio vulnificus, Vibrio alginolyticus, Yersinia enterocolitica, Yersinia pestis, Yersinia pseudotuberculosis, Chlamydophila pneumoniae, Chlamydophila trachomatis, Ricketsia prowazekii, Coxiella burnetii, Ehrlichia chaffeensis, Bartonella hensenae, or the like or combinations thereof.

Dosages and administration of compounds, compositions and formulations of the present disclosure may be determined by one of ordinary skill in the art of clinical pharmacology or pharmacokinetics. An effective amount of the active agent of the present disclosure to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect.

The term “subject” is intended to broadly refer to any animal, such as a mammal, and including humans. Exemplary subjects include but are not limited to humans and non-human primates. The term “subject” as used herein also includes patients and non-patients. The term “patient” refers to individuals suffering or are likely to suffer from a medical condition such as infectious/contagious diseases, viral infections (i.e. diseases caused by virus), bacterial infections (i.e. diseases caused by bacteria), fungal infections (i.e. diseases caused by fungi), parasitic infections (i.e. diseases caused by parasite), respiratory diseases, or the like or combinations thereof, while “non-patients” refer to individuals not suffering and are likely to not suffer from the medical condition. “Non-patients” include healthy individuals, non-diseased individuals and/or an individual free from the medical condition. As used herein, the term “mammal” includes vertebrate such as a human or a large veterinary mammal (e.g., horses, cattle, deer, sheep, llamas, goats, pigs).

The term “bond” refers to a linkage between atoms in a compound or molecule. The bond may be a single bond, a double bond, or a triple bond.

In the definitions of a number of substituents below, it is stated that “the group may be a terminal group or a bridging group”. This is intended to signify that the use of the term is intended to encompass the situation where the group is a terminal group/moiety as well as the situation where the group is a linker between two other portions of the molecule. Using the term “alkyl” having 1 carbon atom as an example, it will be appreciated that when existing as a terminal group, the term “alkyl” having 1 carbon atom may mean —CH₃ and when existing as a bridging group, the term “alkyl” having 1 carbon atom may mean —CH₂- or the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “cyclic” as used herein broadly refers to a structure where one or more series of atoms are connected to form at least one ring. The term includes, but is not limited to, both saturated and unsaturated 5-membered and saturated and unsaturated 6-membered rings. Examples of groups having a cyclic structure include, but are not limited to, cyclopentane, cyclopentene, cyclohexane, cyclohexene, benzene and the like. The term “cyclic” as used herein includes “heterocyclic”.

The term “heterocyclic” as used herein broadly refers to a structure where two or more different kinds of atoms are connected to form at least one ring. For example, a heterocyclic ring may be formed by carbon atoms and at least another atom (i.e. heteroatom) selected from oxygen (O), nitrogen (N) or (NR) and sulfur(S), where R is independently a hydrogen or an organic group. The term also includes, but is not limited to, saturated and unsaturated 5-membered, and saturated and unsaturated 6-membered rings. Examples of groups having a heterocyclic structure include, but are not limited to furan, thiophene, 1H-pyrrole, 2H-pyrrole, 1-pyrroline, 2-pyrroline, 3-pyrroline, 1-pyrazoline, 2-pyrazoline, 3-pyrazoline, 2-imidazoline, 3-imidazoline, 4-imidazoline, pyrazole, imidazole, oxazole, isoxazole, thiazole, isothiazole, 1,2,3-triazole, 1,2,4-triazole, 1,2,3-oxadiazole, disubstituted 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, tetrahydrofuran, tetrahydrothiophene, pyrrolidine, 1,3-dioxolane, 1,2-oxathiolane, 1,3-oxathiolane, pyrazolidine, imidazolidine, pyridine, pyridazine, pyrimidine, pyrazine, 1,2-oxazine, 1,3-oxazine, 1,4-oxazine, thiazine, 1,2,3-triazine, 1,2,4-triazine, 1,3,5-triazine, 2H-pyran, 4H-pyran, 2-pyrone, 4-pyrone, 1,4-dioxin, 2H-thiopyran, 4H-thiopyran, tetrahydropyran, thiane, piperidine, 1,4-dioxane, 1,2-dithiane, 1,3-dithiane, 1,4-dithiane, 1,3,5-trithiane, piperazine, morpholine, thiomorpholine and the like.

The term “amine group” or the like is intended to broadly refer to a group containing —NR², where R is independently a hydrogen or an organic group. The group may be a terminal group or a bridging group.

The term “amide group” or the like is intended to broadly refer to a group containing —C(═O)NR², where R is independently a hydrogen or an organic group. The group may be a terminal group or a bridging group.

The term “aryl” as a group or part of a group denotes (i) an optionally substituted monocyclic, or fused polycyclic, aromatic carbocycle (ring structure having ring atoms that are all carbon) preferably having from 5 to 20, or 5, 6, 7, 8, 9, 10, 11, 12, 13, 14, 15, 16, 17, 18, 19 or 20 carbon atoms per ring. Examples of aryl groups include but are not limited to phenyl, tolyl, xylyl, naphthyl, anthracenyl, phenanthrenyl, fluorenyl, indenyl or indanyl and the like.

The term “heteroaryl” as a group or part of a group refers to groups containing an aromatic ring (preferably a 5- or 6-membered aromatic ring) having one or more carbon atoms (for example 1 to 6 carbon atoms) in the ring replaced by a heteroatom. Suitable heteroatoms may include nitrogen (N) or (NH), oxygen (O) and sulfur(S). Examples of heteroaryl include but are not limited to thiophene, benzothiophene, benzofuran, benzimidazole, benzoxazole, benzothiazole, benzisothiazole, naphtha[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenantridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl and the like. The group may be a terminal group or a bridging group.

The term “halogen” represents chlorine, fluorine, bromine or iodine. The term “halide” represents chloride, fluoride, bromide or iodide.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

It will also be appreciated that where priority is claimed to an earlier application, the full contents of the earlier application is also taken to form part of the present disclosure and may serve as support for embodiments disclosed herein.

### DESCRIPTION OF EMBODIMENTS

Exemplary, non-limiting embodiments of a compound for preparing lipid nanoparticles encapsulating an agent, a method of preparing said compound, a nanoparticle composition comprising said compound and related methods/uses thereto are disclosed hereinafter.

### Compound

There is provided a compound for preparing lipid nanoparticles. In various embodiments, the compound comprises one or more amino acid(s) and/or amino acid residue(s) that is/are ionizable and/or capable of being ionized. In various embodiments, the amino acid and/or amino acid residue comprises one or more carboxylic acid group(s) that is/are ionizable and/or capable of being ionized. In various embodiments therefore, the compound is ionizable and/or capable of being ionized and/or exists in an ionized form at e.g. physiological pH. Advantageously, the ionizable property of the compound (due to presence of ionizable carboxylic acid group) allows for embodiments of the compound to be used as an encapsulation/loading agent and/or delivery vehicle/system. In various embodiments, the compound is designed/configured to allow loading/encapsulation of one or more types of cationic molecules or cationic cargoes. In various embodiments, the compound is also designed/configured to allow the loaded/encapsulated cationic agent to be released from said compound and/or subsequently delivered to a desired target (e.g., cell, cytosol, tissue or organ). The cationic molecules/cargoes to be loaded/encapsulated onto/into/within the compound may include but is not limited to a cationic therapeutic agent, a cationic prophylactic agent or the like. In various embodiments, the cationic molecules/cargoes to be loaded/encapsulated comprises cationic therapeutics. For example, the cationic molecules/cargoes to be loaded/encapsulated may be cationic therapeutics selected from cationic anti-microbial agents, cationic anti-bacterial agents, cationic anti-fungal agents, cationic anti-viral agents, cationic anti-parasitic agents or the like or combinations thereof. In various embodiments, the cationic molecules/cargoes to be loaded/encapsulated comprises cationic macromolecules. For example, the cationic molecules/cargoes to be loaded/encapsulated may be cationic macromolecules selected from cationic peptides, cationic cyclopeptides, cationic drug molecules, or the like or combinations thereof. In various embodiments, the cationic molecules/cargoes to be loaded/encapsulated comprise cationic antibiotics, cationic antivirals, cationic antifungals, cationic antiparasitic or the like or combinations thereof. For example, the molecules/cargoes to be loaded/encapsulated may be polymyxin, polymyxin b, the like or combinations thereof. Advantageously, the compound is suitable for use in encapsulating and/or delivering one or more cationic therapeutic agent and/or cationic prophylactic agent to a desired target (e.g., subject, cell, cytosol, tissue or organ). Accordingly, in various embodiments, there is also provided a carrier, nanocarrier or delivery system/vehicle comprising the compound or its ionized form thereof.

Advantageously, the compound is designed/configured to be ionizable at about physiological pH (or about neutral pH). In various embodiments, the compound is capable of being ionized at physiological pH range of from about 7.00 to about 7.80, from about 7.05 to about 7.75, from about 7.10 to about 7.70, from about 7.15 to about 7.65, from about 7.20 to about 7.60, from about 7.25 to about 7.55, from about 7.30 to about 7.50, from about 7.35 to about 7.45, about 7.36, about 7.37, about 7.38, about 7.39, about 7.40, about 7.41, about 7.42, about 7.43, about 7.44, or about 7.45.

In various embodiments, the compound comprises a structure that is represented by general formula (1) or an ionized form thereof:

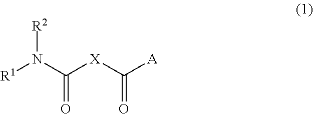

wherein
R¹ and R² are each independently a hydrophobic tail/chain/group or contains at least linear aliphatic, branched aliphatic and/or cyclic hydrocarbons;
X comprises an optionally substituted linear aliphatic, optionally substituted branched aliphatic and/or optionally substituted cyclic hydrocarbons; and
A comprises a short anionic amino acid sequence/anionic oligopeptide sequence (1-5 amino acids).

In various embodiments, A comprises one or more amino acid(s) and/or amino acid residue group(s) that is/are ionizable and/or capable of being ionized. In various embodiments, A comprises an amino acid sequence of 1, 2, 3, 4, or 5 anionic amino acid residues. It will be appreciated that a short amino acid sequence of no more than 5 anionic amino acids is advantageous as long amino acid sequences are difficult to synthesize and may not work well with antibiotics that typically do not contain many positive charges. The amino acid residues may be selected from glutamic acid (or glutamate), aspartic acid (or aspartate), or combinations thereof. In various embodiments, when A comprises 5 glutamic acids (or glutamates), an effective antibiotic delivery may be achieved.

In various embodiments, A is capable of being ionized at physiological pH range of from about 7.00 to about 7.80. In various embodiments, A may be oligoglutamate (1-5 amino acids) or oligo (glutamic acid) (1-5 amino acids). For example, A may comprise oligo (glutamate) 5. In various embodiments, the term “oligoglutamates” may comprise and/or may be used interchangeably with the terms “oligo (glutamate) 5”, “oligo (glutamic acid) 5”, “oligo (glutamic acid) 5”, “OGA”, “oligo-α-glutamic acid₅”, “oligo-α-glutamates”, “a-OGA”, “oligo-α-L-glutamic acids”, “oligo-α-L-glutamates”, “oligo-γ-glutamic acid₅”, “oligo-γ-glutamates”, “γ-OGA”, or the like or enantiomers thereof.

In various embodiments, the terms “cationic” and “anionic” are used with reference to the charge of the compound under physiological pH conditions.

In various embodiments, the compound comprises a lipid compound. In various embodiments, the compound comprises hydrophobic parts/tails/chains/groups at both R¹ and R². In various embodiments, the hydrophobic tail/chain/group at R¹ and R² each independently comprises optionally substituted alkyl. The alkyl may have at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 carbon atoms. For example, R¹ and R² may be each independently C_{y}H_{2y+1}, where y≥5, y≥6, y≥7, y≥8, y≥9, y≥10, y≥11, y≥12, y≥13, y≥14, y≥15, y≥16, y≥17, y≥18, y≥19, y≥20, y≥21, y≥22, y≥23 or y≥24. Advantageously, in various embodiments, the presence of hydrophobic parts/tails/chains/groups in the compound aids in imparting improved cellular uptake, thereby leading to a higher and/or better antimicrobial activity (e.g., antibacterial activity) displayed by embodiments of the compound.

In various embodiments, the compound is amphiphilic/amphipathic and comprises hydrophilic and hydrophobic parts. In various embodiments, the compound comprises hydrophilic part(s) at A (e.g., carboxylic acid group(s)). In various embodiments, the compound comprises hydrophobic parts/tails/chains/groups at both R¹ and R².

In various embodiments, X comprises optionally substituted alkyl, optionally substituted alkenyl, optionally substituted alkynyl or the like or combinations thereof. The alkyl, alkenyl or alkynyl may have at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least carbon atoms. For example, X may be independently C₂H_{2z}, where z≥1, z≥2, z≥3, z≥4, z≥5, y≥6, y≥7, y≥8, y≥9, y≥10, y≥11, y≥12, y≥13, y≥14, y≥15, y≥16, y≥17, y≥18, y≥19, y≥20, y≥21, y≥22, y≥23, y≥24, y≥25, y≥26, y≥27, y≥28, y≥29, or y≥30. In various embodiments, X is selected from methyl, ethyl, n-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, or the like or combinations thereof.

In various embodiments, X is optionally substituted alkyl having from 1 to carbon atoms. For example, X may be —(CR^{3′}R^{4′})ₘ—, where m may be from 1 to 5.

In various embodiments when A comprises oligoglutamate (1-5 amino acids), the compound comprises a structure that is represented by general formula (1A) or an ionized form thereof:

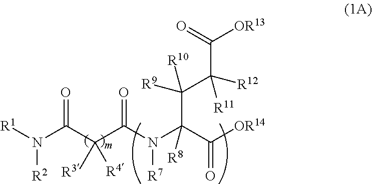

wherein
R^{3′}, R^{4′}, R⁷ to R¹⁴ are each independently selected from the group consisting of H, optionally substituted alkyl, optionally substituted alkenyl, optionally substituted alkynyl, and combinations thereof;
m is 1, 2, 3, 4 or 5; and
n is 1, 2, 3, 4 or 5.

In various embodiments, X is optionally substituted alkyl having 2 carbon atoms. For example, the compound may be derived from succinic anhydride. For example, m may be 2 and X may be —CR³R⁴CR⁵R⁶—.

In various embodiments when A comprises oligoglutamate (1-5 amino acids), the compound comprises a structure that is represented by general formula (1B) or an ionized form thereof:

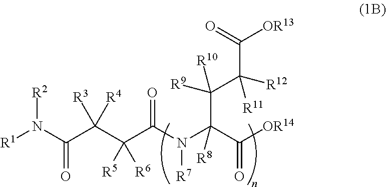

wherein
R³ to R¹⁴ are each independently selected from the group consisting of H, optionally substituted alkyl, optionally substituted alkenyl, optionally substituted alkynyl, and combinations thereof; and
n is 1-5.

In various embodiments, R¹ and R² contain one or more features and/or share one or more properties that are similar to those described above (e.g., as defined in general formula (1)).

In various embodiments, n is an integer of 1-5. In various embodiments, n is 1, 2, 3, 4 or 5.

In various embodiments, R³ to R¹⁴ are each independently selected from H, optionally substituted alkyl, optionally substituted alkenyl or optionally substituted alkynyl. For example, R³ to R¹⁴ may be selected from methyl, ethyl, n-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, or the like or combinations thereof.

In various embodiments, R¹³ is H. In such embodiments, —C(═O)OR¹³ is —C(═O)OH (e.g., an ionizable carboxylic acid group).

In various embodiments, R¹⁴ is H. In such embodiments, —C(═O)OR¹⁴ is —C(═O)OH (e.g., an ionizable carboxylic acid group).

In various embodiments, the compound is in an ionized form, where —C(═O)OR¹³ and/or —C(═O)OR¹⁴ has/have been ionized to become a negatively charged group. For example, the compound is an ionized form of general formula (1), ionized form of general formula (1A) or ionized form of general formula (1B). In various embodiments, —C(═O)OR¹³ and/or —C(═O)OR¹⁴ is ionized/deprotonated at about physiological pH (or about neutral pH) to become a negatively charged group/ion/cation. In various embodiments, —C(═O)OR¹³ and/or —C(═O)OR¹⁴ is/are deprotonated to become —C(═O)O—.

In various embodiments, the term “compound” may comprise and/or may be used interchangeably with the terms “lipid”, “lipid compound”, “ionizable lipid”, “ionizable lipid compound”, “anionic lipid compound”, “ionizable anionic lipid compound” or the like.

In various embodiments, the compound comprises 1, 2, 3, 4, or 5 ionizable carboxylic acid groups.

In various embodiments, the compound has an average or mean molecular weight of from about 400.0 g/mol to about 1,500.0 g/mol, or from about 600.0 g/mol to about 2,500.0 g/mol.

In various embodiments, the compound comprises the following structure:

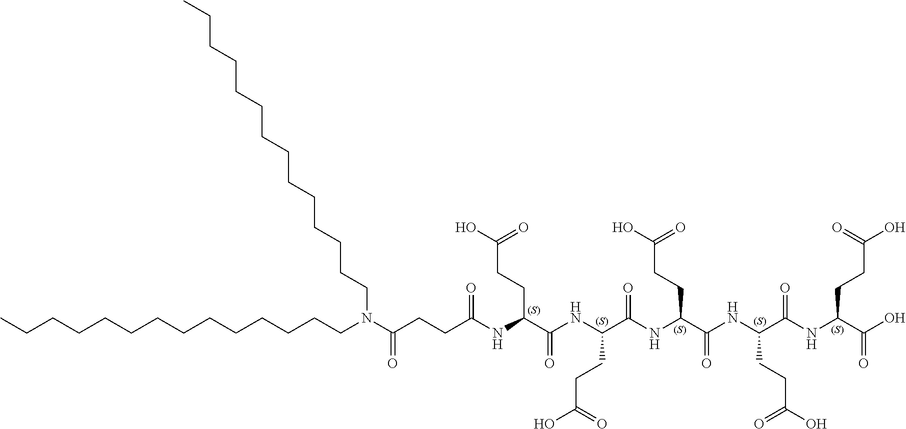

### Method of Preparing Compound

There is provided a method of preparing a compound represented by general formula (1), (1A) or (1B) as disclosed herein, the method comprising:
(a-i) reacting a resin with a fully protected (i.e. N- and C-protected) amino acid to attach the fully protected amino acid to the resin;
(a-ii) removing N-protecting group from the fully protected amino acid to obtain a partially protected (i.e. C-protected) amino acid;
(a-iii) reacting the partially protected (i.e. C-protected) amino acid with another fully protected amino acid to attach the fully protected amino acid to the partially protected amino acid via the N-terminus to obtain a resin-supported fully protected peptide represented by general formula (2):

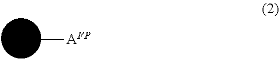

wherein
● represents resin; and
A^{FP} represents fully protected peptide (e.g., oligopeptide);
(a-iv) removing N-protecting group from the resin-supported fully protected peptide represented by general formula (2) to obtain a resin-supported partially protected (i.e. C-protected) peptide represented by general formula (3):

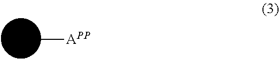

wherein
● represents resin; and
A^{PP} represents partially protected peptide (e.g., oligopeptide);
(a-v) reacting the resin-supported partially protected (i.e. C-protected) peptide represented by general formula (3) with an acid anhydride represented by general formula (4) to obtain a first intermediate compound represented by general formula (5);

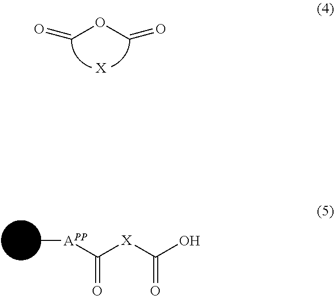

(a-vi) reacting the first intermediate compound represented by general formula (5) with an amine compound represented by general formula (6) to obtain a second intermediate represented by general formula (7); and

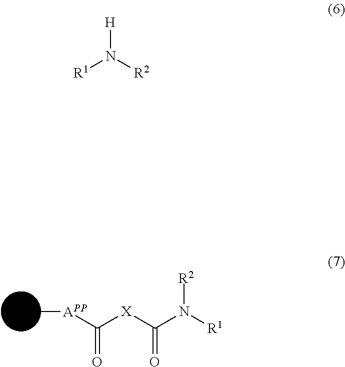

(a-vii) removing the resin from the second intermediate represented by general formula (7) to obtain a compound represented by general formula (1); and

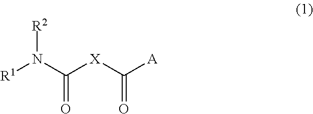

(a-viii) optionally ionizing A to become a negatively charged group.

In various embodiments, R¹, R², X and/or A contain one or more features and/or share one or more properties that are similar to those described above (e.g., as defined in general formula (1)).

In various embodiments, the method further comprises optionally repeating step (a-ii) and/or step (a-iii) until an oligopeptide of a desired length is obtained.

Advantageously, embodiments of the method are straightforward to perform and have a low production/manufacturing cost (i.e. cost effective) as they may be carried out simply in few synthetic/reaction steps. Advantageously, embodiments of the method are scalable and/or have substantially high scalability. Advantageously, embodiments of the method are capable of synthesizing such anionic oligoglutamate lipids rapidly, which is uniquely different from known solid phase peptide synthesis methods.

In various embodiments, the method is carried out in solid phase and/or in the presence of a solid support. In various embodiments, the method comprises solid phase synthesis. In various embodiments, the resin is an acid sensitive resin. For example, the resin may be 2-chlorotrityl chloride resin, Wang resin (i.e., p-Alkoxy-benzyl alcohol polymer-bound, p-Alkoxybenzyl alcohol resin, or [4-(Hydroxymethyl)phenoxymethyl]polystyrene), or the like. It will be appreciated that any resin that effectively serves as a support for the synthesis of protected peptide by e.g., Fmoc strategy may be used in embodiments of the method disclosed herein. In various embodiments, when the resin is Wang resin, the method above may be carried out as described with the exception that the final acidic cleavage step may use a solvent comprising 95% Trifluoroacetic acid/Dichloromethane.

In various embodiments, the N-protecting groups comprise 9-fluorenylmethyloxycarbonyl (Fmoc), tert-butoxycarbonyl (Boc), N-carboxybenzyl or benzyloxycarbonyl, the like or combinations thereof.

In various embodiments, the steps (a-ii) and/or (a-iv) comprise performing deprotection to remove the N-protecting groups. In various embodiments, deprotection is performed in the presence of a base. The base may be piperidine, 1,8-diazabicyclo[5.4.0]undec-7-ene (DBU), or the like.

In various embodiments, the reacting steps (a-i), (a-iii), (a-v) and/or (a-vi) is/are performed in the presence of an organic base. The organic base may be a poorly nucleophilic or non-nucleophilic base, e.g., sterically hindered amine. In various embodiments, the organic base is N,N-diisopropylethylamine (DIPEA), triethylamine (TEA), Hünig's base, or the like.

In various embodiments, the acid anhydride represented by general formula (4) comprises cyclic anhydride. The cyclic anhydride may be succinic anhydride, oxalic anhydride, malonic anhydride, glutaric anhydride, adipic anhydride, pimelic anhydride, or the like.

In various embodiments, the step (viii) of removing the resin comprises subjecting the second intermediate represented by general formula (7) to acidic conditions. For example, the step (viii) may be carried out in the presence of one or more acids (e.g., trifluoroacetic acid (TFA), hydrobromic acid (HBr), acetic acid (AcOH) or the like or combinations thereof).

In various embodiments, the steps (a-i), (a-ii), (a-iii), (a-iv), (a-v), (a-vi) and/or (a-vii) comprises one or more of the following steps: dispersing, mixing, stirring, dissolving, sonicating and/or ultrasonicating.

In various embodiments, the steps (a-i), (a-ii), (a-iii), (a-iv), (a-v), (a-vi) and/or (a-vii) is/are performed in the presence of an organic solvent. In various embodiments, any organic solvent that effectively serves as a medium to contain the components of the reaction mixture (e.g., reactants/substrates) may be used in embodiments of the reaction mixture disclosed herein. In various embodiments, the organic solvent is capable of substantially dissolving the components present in the reaction mixture. The organic solvent may comprise ethanol, isopropanol, acetonitrile, ethyl acetate, methanol, tetrahydrofuran, dimethyl sulfoxide, dimethylformamide, dichloromethane, N-methyl-2-pyrrolidone (NMP), or the like or combinations thereof.

In various embodiments, the steps (a-i), (a-ii), (a-iii), (a-iv), (a-v), (a-vi) and/or (a-vii) is/are performed over a time duration of from about 1 hour to about 72 hours, from about 2 hours to about 60 hours, from about 3 hours to about 48 hours, from about 4 hours to about 36 hours, from about 5 hours to about 24 hours, or from about 6 hours to about 12 hours.

In various embodiments, the steps (a-i), (a-ii), (a-iii), (a-iv), (a-v), (a-vi) and/or (a-vii) are performed at room temperature e.g., from about 20° C. to about 30° C., about 21° C., about 22° C., about 23° C., about 24° C., about 25° C., about 26° C., about 27° C., about 28° C., about 29° C., or about 30° C.

In various embodiments, the steps (a-i), (a-ii), (a-iii), (a-iv), (a-v), (a-vi) and/or (a-vii) are optionally performed with heating at a temperature e.g., from about 50° C. to about 150° C., from about 60° C. to about 140° C., from about 70° C. to about 130° C., from about 80° C. to about 120° C., from about 90° C. to about 110° C., or about 100° C.

In various embodiments, the method further comprises:
(b-i) a step of isolating the resin-supported fully protected amino acid after step (a-i)
(b-ii) a step of isolating the resin-supported partially protected amino acid after step (a-ii);
(b-iii) a step of isolating the resin-supported fully protected peptide represented by general formula (2) after step (a-iii);
(b-iv) a step of isolating the resin-supported partially protected peptide represented by general formula (3) after step (a-iv);
(b-v) a step of isolating the first intermediate compound represented by general formula (5) after step (a-v);
(b-vi) a step of isolating the second intermediate compound represented by general formula (7) after step (a-vi); and
(b-vii) a step of isolating the compound represented by general formula (1) after step (a-vii).

In various embodiments, the isolating step(s) comprises one or more of the following steps: re-dissolving, purifying, centrifuging, quenching, washing, filtering, precipitating and/or recrystallizing the resin-supported fully protected amino acid, resin-supported partially protected amino acid, the resin-supported fully protected peptide represented by general formula (2), the resin-supported partially protected peptide represented by general formula (3), the first intermediate compound represented by general formula (5), the second intermediate compound represented by general formula (7) and/or the compound represented by general formula (1). The step(s) of purifying, centrifuging, quenching and/or washing may be repeated at least 1 time, at least 2 times, at least 3 times, at least 4 times, at least 5 times, at least 6 times, at least 7 times, at least 8 times, at least 9 times, at least 10 times, at least 15 times, at least 20 times with a washing medium. In various embodiments, the isolating step is performed to remove by-products and/or unreacted reagents after each of steps (a-i), (a-ii), (a-iii), (a-iv), (a-v), (a-vi) and/or (a-vii). In various embodiments, the washing medium comprises organic solvent such as ethanol, isopropanol, acetonitrile, ethyl acetate, methanol, tetrahydrofuran, dimethyl sulfoxide, dimethylformamide, dichloromethane, N-methyl-2-pyrrolidone (NMP), or the like or combinations thereof.

In various embodiments, the method further comprises one or more of the following post reaction steps: drying the compound represented by general formula (1), optionally under low temperature (e.g., freeze drying or lyophilizing) and/or under vacuum. The step(s) of drying may be performed in the presence of an inert gas such as argon or nitrogen or in the absence of reactive gases such as oxygen (e.g., dissolved oxygen).

In various embodiments, step (a-viii) is present and is performed at physiological pH range of from about 7.00 to about 7.80, from about 7.05 to about 7.75, from about 7.10 to about 7.70, from about 7.15 to about 7.65, from about 7.20 to about 7.60, from about 7.25 to about 7.55, from about 7.30 to about 7.50, from about 7.35 to about 7.45, about 7.36, about 7.37, about 7.38, about 7.39, about 7.40, about 7.41, about 7.42, about 7.43, about 7.44, or about 7.45.

### Nanoparticle Composition

Advantageously, in various embodiments, the ionizable property of the compound represented by general formula (1) (due to presence of ionizable carboxylic acid group) allows for the condensation and encapsulation/loading of cationic molecules/cargoes into embodiments of the compound through electrostatic interaction, thereby forming nanoparticles in a composition. In various embodiments, embodiments of the compound are capable of forming nanoparticles in a composition. In various embodiments, in the presence of a composition comprising cationic molecules/cargoes (e.g., cationic therapeutic agent and/or cationic prophylactic agent), the one or more carboxylic acid group(s) in the compound (e.g., ionized/deprotonated carboxylic acid group(s) in the lipid compound) condenses and encapsulates/loads the molecules/cargoes into the compound to form nanoparticles (e.g., lipid nanoparticles (LNPs)) in the composition.

The term “nanoparticles” may comprise and/or may be used interchangeably with the terms “lipid nanoparticles”, “encapsulated lipid nanoparticles”, “loaded lipid nanoparticles”, “LNPs” or the like.

There is provided a nanoparticle composition comprising:
(i) a compound represented by general formula (1), general formula (1A) or general formula (1B) and/or ionized form thereof as disclosed herein; and
(ii) a cationic therapeutic agent and/or cationic prophylactic agent that is encapsulated/loaded in the compound represented by general formula (1), general formula (1A) or general formula (1B) and/or ionized form thereof.

In various embodiments, the compound is deprotonated to form a negatively charged or anionic compound. In various embodiments, the molecules/cargoes to be loaded/encapsulated (or therapeutic agent and/or prophylactic agent) comprises positively charged or cationic molecules/cargoes, e.g., cationic therapeutics, cationic antimicrobials, cationic macromolecules or the like or combinations thereof. For example, the therapeutic agent and/or prophylactic agent may be cationic antibiotic agent, cationic antiviral agent, cationic antifungal agent, cationic antiparasitic agent or the like or combinations thereof. Advantageously, in various embodiments, the compound represented by general formula (1) is capable of being ionized (e.g., deprotonated) at physiological pH (or neutral pH) such that the composition encapsulates a cationic therapeutic and/or is cationic prophylactic agent that coupled/bonded/linked/bound to the composition/nanoparticles. The cationic therapeutic and/or cationic prophylactic agent may be coupled/bonded/linked/bound to the composition/nanoparticles via electrostatic interaction and/or other physical interactions. In various embodiments, the cationic therapeutic and/or cationic prophylactic agent is electrostatically and/or physically coupled/bonded/linked/bound to the composition/nanoparticle.

In various embodiments, the compounds (e.g., lipid compounds) are deprotonated readily in water, leading to a negatively charged compounds that condense molecules/cargoes (e.g., positively charged therapeutic and/or prophylactic agent) into lipid nanoparticles (LNPs) through electrostatic and/or physical interaction, forming encapsulated LNPs. For example, the anionic lipid nanoparticles encapsulating polymyxin B may adhere to the surface of the outer membrane of Gram-negative bacteria, where the delivery of polymyxin B may happen and consequently be used for treatment purposes.

Advantageously, the composition is suitable for use in the encapsulation, and/or delivery of one or more cationic therapeutic agent and/or cationic prophylactic agent e.g., to a desired target (such as subject, cell, cytosol, tissue or organ).

In various embodiments, the composition further comprises:
(a) neutral/helper lipid;
(b) sterol; and
(c) polyethylene glycol (PEG)-modified lipid.

The term “polyethylene glycol (PEG)-modified lipid” may comprise and/or may be used interchangeably with the terms “PEGylated lipid” and “lipid modified with PEG”.

In various embodiments, the compound or its ionized form thereof, neutral/helper lipid, sterol, and PEG-modified lipid are mixed/dissolved in an organic solvent. In various embodiments, any organic solvent that effectively serves as a medium to contain the components of the reaction mixture (e.g., reactants/substrates) may be used in embodiments of the reaction mixture disclosed herein. In various embodiments, the organic solvent is capable of substantially dissolving the components present in the reaction mixture. The organic solvent may comprise ethanol, isopropanol, acetonitrile, ethyl acetate, methanol, tetrahydrofuran, dimethyl sulfoxide, dimethylformamide or the like or combinations thereof.

In various embodiments, the cationic therapeutic and/or cationic prophylactic agent, helper lipid, sterol, compound represented by general formula (1), and PEG-modified lipid are mixed/dissolved at a molar/weight ratio of about 10-120:about 1-30:about 10-150:about 1-30:about 0.5-10. For example, the compound or its ionized form thereof, neutral/helper lipid, sterol, and PEG-modified lipid may be mixed. In various embodiments, the compound represented by general formula (1) or its ionized form thereof, neutral/helper lipid, sterol, and PEG-modified lipid are mixed/dissolved at a molar ratio of about 37:9:43:8:3, or 38:9:43:8:2.

In various embodiments, the composition comprises from about 10.0 mol % to about 120.0 mol %, from about 15.0 mol % to about 115.0 mol %, from about 20.0 mol % to about 110.0 mol %, from about 25.0 mol % to about 105.0 mol %, from about 30.0 mol % to about 100.0 mol %, from about 35.0 mol % to about 95.0 mol %, from about 40.0 mol % to about 90.0 mol %, from about 45.0 mol % to about 85.0 mol %, from about 50.0 mol % to about 80.0 mol %, from about 55.0 mol % to about 75.0 mol %, from about 60.0 mol % to about 70.0 mol %, or about 65.0 mol % of the cationic therapeutic agent and/or cationic prophylactic agent.

In various embodiments, the neutral/helper lipid comprises a phospholipid such as an unsaturated lipid. Examples of phospholipid includes, but are not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and the like and combinations thereof.

In various embodiments, the composition comprises from about 1.0 mol % to about 30.0 mol %, from about 2.0 mol % to about 29.0 mol %, from about 3.0 mol % to about 28.0 mol %, from about 4.0 mol % to about 27.0 mol %, from about 5.0 mol % to about 26.0 mol %, from about 6.0 mol % to about 25.0 mol %, from about 7.0 mol % to about 24.0 mol %, from about 8.0 mol % to about 23.0 mol %, from about 9.0 mol % to about 22.0 mol %, from about 10.0 mol % to about 21.0 mol %, from about 11.0 mol % to about 20.0 mol %, from about 12.0 mol % to about 19.0 mol %, from about 13.0 mol % to about 18.0 mol %, from about 14.0 mol % to about 17.0 mol %, or from about 15.0 mol % to about 16.0 mol % of neutral/helper lipid.

In various embodiments, the sterol is selected from cholesterol, fecosterol, sitosterol, ergosterol, campesterol, stigmasterol, brassicasterol, avenasterol, or the like or combinations thereof.

In various embodiments, the composition comprises from about 10.0 mol % to about 150.0 mol %, from about 15.0 mol % to about 145.0 mol %, from about 20.0 mol % to about 140.0 mol %, from about 25.0 mol % to about 135.0 mol %, from about 30.0 mol % to about 130.0 mol %, from about 35.0 mol % to about 125.0 mol %, from about 40.0 mol % to about 120.0 mol %, from about 45.0 mol % to about 115.0 mol %, from about 50.0 mol % to about 110.0 mol %, from about 55.0 mol % to about 105.0 mol %, from about 60.0 mol % to about 100.0 mol %, from about 65.0 mol % to about 95.0 mol %, from about 70.0 mol % to about 90.0 mol %, from about 75.0 mol % to about 85.0 mol %, or about 80.0 mol % of sterol.

In various embodiments, the composition comprises from about 1.0 mol % to about 30.0 mol %, from about 2.0 mol % to about 29.0 mol %, from about 3.0 mol % to about 28.0 mol %, from about 4.0 mol % to about 27.0 mol %, from about 5.0 mol % to about 26.0 mol %, from about 6.0 mol % to about 25.0 mol %, from about 7.0 mol % to about 24.0 mol %, from about 8.0 mol % to about 23.0 mol %, from about 9.0 mol % to about 22.0 mol %, from about 10.0 mol % to about 21.0 mol %, from about 11.0 mol % to about 20.0 mol %, from about 12.0 mol % to about 19.0 mol %, from about 13.0 mol % to about 18.0 mol %, from about 14.0 mol % to about 17.0 mol %, or from about 15.0 mol % to about 16.0 mol % of compound represented by general formula (1).

In various embodiments, the PEG-modified lipid is selected from PEG-modified phosphatidylethanolamines, PEG-modified phosphatidic acids, PEG-modified ceramides, PEG-modified dialkylamines, PEG-modified diacylglycerols, PEG-modified dialkylglycerols, or the like or combinations thereof. Examples of PEG-modified/PEGylated lipid include, but is not limited to, 2-[(polyethylene glycol)-2000]-N,N-ditetradecylacetamide (ALC-0159), R-3-[(w-methoxy-poly(ethylene glycol) 2000) carbamoyl]-1,2-dimyristyloxlpropyl-3-amine (PEG-c-DOMG), 3-N-[(w-methoxypoly(ethyleneglycol) 2000) carbamoyl]-1,2-dimyristyloxy-propylamine (PEG-S-DMG), PEG-DMPE (1,2-dimyristoyl-sn-glycero-3-phosphoethanolamine-N-[(polyethylene glycol)-methoxy](sodium salt)), PEG-DPPC, PEG-DSPE lipid, 14:0 PEG2000 PE (1,2-dimyristoyl-sn-glycero-3-phosphoethanolamine-N-[methoxy (polyethylene glycol)-2000](ammonium salt)), 18:0 PEG2000 PE (1,2-distearoyl-sn-glycero-3-phosphoethanolamine-N-[methoxy (polyethylene glycol)-2000](ammonium salt)), or the like or combinations thereof.

In various embodiments, the composition comprises from about 0.5 mol % to about 10.0 mol %, from about 1.0 mol % to about 9.5 mol %, from about 1.5 mol % to about 9.0 mol %, from about 2.0 mol % to about 8.5 mol %, from about 2.5 mol % to about 8.0 mol %, from about 3.0 mol % to about 7.5 mol %, from about 3.5 mol % to about 7.0 mol %, from about 4.0 mol % to about 6.5 mol %, from about 4.5 mol % to about 6.0 mol %, or from about 5.0 mol % to about 5.5 mol % of PEG-modified lipid.

In various embodiments, the cationic therapeutic agent and/or cationic prophylactic agent is provided in an aqueous composition. The aqueous composition may be deionized, distilled and/or filtered water. In various embodiments, the aqueous composition is substantially devoid of components or contaminants that may react unfavourably with the cationic therapeutic and/or cationic prophylactic agent. For example, the aqueous composition is deionized water, normal saline/saline (0.9%, pH 5.5), phosphate-buffered saline (PBS), PBS (pH 7.4), tris-buffered saline, sodium acetate buffer, sodium citrate buffer, MilliQ water, or the like or combinations thereof.

In various embodiments, the nanoparticle composition comprises nanoparticles formed from the compound represented by general formula (1), general formula (1A) or general formula (1B) and/or ionized form thereof.

### Nanoparticles

There is provided nanoparticles (e.g., lipid nanoparticles) comprising:
(i) the compound represented by general formula (1), general formula (1A) or general formula (1B) and/or ionized form thereof as disclosed herein; and
(ii) a cationic therapeutic and/or cationic prophylactic agent that is encapsulated/loaded/coupled/bonded/linked/bound in/to said compound represented by general formula (1) or ionized form thereof.

In various embodiments, the nanoparticles have a charge ratio of (ii):(i) that is from about 1:1 to about 40:1. In various embodiments, the charge ratio of the cationic therapeutic and/or cationic prophylactic agent that is encapsulated to the compound represented by general formula (1), general formula (1A) or general formula (1B) and/or ionized form thereof as disclosed herein is from about 1:1 to about 40:1, from about 2:1 to about 39:1, from about 3:1 to about 38:1, from about 4:1 to about 37:1, from about 5:1 to about 36:1, from about 6:1 to about 35:1, from about 7:1 to about 34:1, from about 8:1 to about 33:1, from about 9:1 to about 32:1, from about 10:1 to about 31:1, from about 11:1 to about 30:1, from about 12:1 to about 29:1, from about 13:1 to about 28:1, from about 14:1 to about 27:1, from about 15:1 to about 26:1, from about 16:1 to about 25:1, from about 17:1 to about 24:1, from about 18:1 to about 23:1, from about 19:1 to about 22:1, or from about 20:1 to about 21:1.

In various embodiments, the encapsulation/loading/binding efficiency/capacity of the cationic therapeutic agent, and/or cationic prophylactic agent in the composition/nanoparticles is at least about 10.0%, at least about 20.0%, at least about 30.0%, at least about 40.0%, at least about 50.0%, at least about 60.0%, at least about 70.0%, at least about 80.0%, at least about 90.0%, at least about 95.0%, at least about 96.0%, at least about 97.0%, at least about 98.0%, at least about 99.0%, at least about 99.5%, or at least about 99.9%.

In various embodiments, the nanoparticles have an average or mean particle size (or diameter) of from about 20.0 nm to about 200.0 nm, from about 30.0 nm to about 190.0 nm, from about 40.0 nm to about 180.0 nm, from about 50.0 nm to about 170.0 nm, from about 60.0 nm to about 160.0 nm, from about 70.0 nm to about 150.0 nm, from about 80.0 nm to about 140.0 nm, from about 90.0 nm to about 130.0 nm, from about 100.0 nm to about 120.0 nm, or about 110.0 nm.

In various embodiments, the composition comprising the nanoparticles has a polydispersity index (PDI) of from about 0.01 to about 0.80, from about 0.0125 to about 0.45, from about 0.015 to about 0.40, from about 0.020 to about 0.35, from about 0.025 to about 0.30, from about 0.030 to about 0.25, from about 0.035 to about 0.20, from about 0.040 to about 0.15, from about 0.045 to about 0.10, from about 0.050 to about 0.095, from about 0.055 to about 0.090, from about 0.060 to about 0.085, from about 0.065 to about 0.080, or from about 0.070 to about 0.075. Advantageously, in various embodiments, the nanoparticles have a narrow particle size distribution and/or the nanoparticles or nanoparticle composition is relatively/substantially homogenous.

In various embodiments, the nanoparticles have a zeta potential of from about −20.0 mV to about +20.0 mV, from about −19.0 mV to about +19.0 mV, from about −18.0 mV to about +18.0 mV, from about −17.0 mV to about +17.0 mV, from about −16.0 mV to about +16.0 mV, from about −15.0 mV to about +15.0 mV, from about −14.0 mV to about +14.0 mV, from about −13.0 mV to about +13.0 mV, from about −12.0 mV to about +12.0 mV, from about −11.0 mV to about +11.0 mV, from about −10.0 mV to about +10.0 mV, from about −9.0 mV to about +9.0 mV, from about −8.0 mV to about +8.0 mV, from about −7.0 mV to about +7.0 mV, from about −6.0 mV to about +6.0 mV, from about −5.0 mV to about +5.0 mV, from about −4.0 mV to about +4.0 mV, from about −3.0 mV to about +3.0 mV, from about −2.0 mV to about +2.0 mV, from about −1.0 mV to about +1.0 mV, or about 0 mV in saline or in a physiological environment (e.g. in normal saline, in a 0.90% w/v NaCl solution or 10% w/v fetal bovine serum-containing saline Advantageously, in various embodiments, the nanoparticles have a substantially neutral surface charge, making the nanoparticles suitable/desirable for in vivo applications.

In various embodiments, the composition comprising the nanoparticles have a colloidal stability of at least about 4 weeks, at least about 3 weeks, at least about 2 weeks, at least about 1 week, at least about 6 days, at least about 5 days, at least about 4 days, at least about 3 days, at least about 2 days, at least about 1 day without an appreciable loss in desired properties. Advantageously, in various embodiments, the composition can be stably stored for up to a week, or up to about 4 weeks without experiencing substantial aggregation and/or agglomeration of nanoparticles. Advantageously, in various embodiments, the hydrodynamic size and/or neutral surface charge of the nanoparticles remains substantially the same (or are substantially unchanged) after a storage duration of at least about a week or at least about 4 weeks.

In various embodiments, the composition comprising the nanoparticles can be stored at a temperature of from about −10° C. to about 10° C., from about-9° C. to about 9° C., from about −8° C. to about 8° C., from about −7° C. to about 7° C., from about −6° C. to about 6° C., from about −5° C. to about 5° C., from about −4° C. to about 4° C., from about −3° C. to about 3° C., from about −2° C. to about 2° C., from about −1° C. to about 1° C., or about 0° C.

In various embodiments, the nanoparticles have a cell viability of at least about 80.0%, at least about 90.0%, at least about 95.0%, at least about 96.0%, at least about 97.0%, at least about 98.0%, at least about 99.0%, at least about 99.5%, or at least about 99.9%.

In various embodiments, the nanoparticles have a minimum bactericidal concentration (MBC) to minimum inhibitory concentration (MIC) ratio that is no more than about 6. In various embodiments, the nanoparticles have a minimum bactericidal concentration (MBC) to minimum inhibitory concentration (MIC) ratio that is no more than about 6, no more than about 5.5, no more than about 5, no more than about 4.5, no more than about 4, no more than about 3.5, no more than about 3, no more than about 2.5, no more than about 2, no more than about 1.5, no more than about 1. The nanoparticles may have a minimum bactericidal concentration (MBC) to minimum inhibitory concentration (MIC) ratio that is about 1, about 1.25, about 1.50, about 1.75, about 2, about 2.25, about 2.50, about 2.75, about 3, about 3.25, about 3.50, about 3.75, or about 4.

In various embodiments, the nanoparticles have a minimum bactericidal concentration (MBC) that is no more than about 6. In various embodiments, the nanoparticles have a minimum bactericidal concentration (MBC) that is no more than about 6, no more than about 5.5, no more than about 5, no more than about 4.5, no more than about 4, no more than about 3.5, no more than about 3, no more than about 2.5, no more than about 2, no more than about 1.5, no more than about 1. The nanoparticles may have a minimum bactericidal concentration (MBC) that is about 1, about 1.25, about 1.50, about 1.75, about 2, about 2.25, about 2.50, about 2.75, about 3, about 3.25, about 3.50, about 3.75, or about 4.

In various embodiments, the nanoparticles have a minimum inhibitory concentration (MIC) ratio that is no more than about 6. In various embodiments, the nanoparticles have a minimum inhibitory concentration (MIC) ratio that is no more than about 6, no more than about 5.5, no more than about 5, no more than about 4.5, no more than about 4, no more than about 3.5, no more than about 3, no more than about 2.5, no more than about 2, no more than about 1.5, no more than about 1. The nanoparticles may have a minimum inhibitory concentration (MIC) ratio that is about 1, about 1.25, about 1.50, about 1.75, about 2, about 2.25, about 2.50, about 2.75, about 3, about 3.25, about 3.50, about 3.75, or about 4.

In various embodiments, the composition/compound/nanoparticles is/are biocompatible, i.e. the composition/compound/nanoparticle is compatible with biological systems or parts of the biological systems without substantially or significantly eliciting an adverse physiological response such as a toxic reaction/response (e.g., cytotoxicity, neurotoxicity, nephrotoxicity or the like), an immune reaction/response, an injury or the like when used on the human or animal body. In various embodiments, the composition/compound/nanoparticle is substantially devoid of substances that elicit an adverse physiological response. Advantageously, the nanoparticles (e.g., lipid nanoparticles) are capable of binding cationic therapeutic agent, and/or cationic prophylactic agent effectively and/or providing high antimicrobial activity (e.g., antibacterial activity) without causing/inducing substantial or any cytotoxicity, neurotoxicity, nephrotoxicity or the like.

### Method of Preparing Nanoparticles

There is provided a method of preparing nanoparticles as disclosed herein, the method comprising:
(c-i) preparing an aqueous composition comprising cationic therapeutic and/or cationic prophylactic agent;
(c-ii) mixing the aqueous composition obtained from (c-i) with the composition as disclosed herein to obtain nanoparticles.

In various embodiments, the step (c-i) comprises mixing the cationic therapeutic and/or cationic prophylactic agent in an aqueous composition. The aqueous composition may be deionized, distilled and/or filtered water. In various embodiments, the aqueous composition is substantially devoid of components or contaminants that may react unfavourably with the cationic therapeutic and/or cationic prophylactic agent. For example, the aqueous composition is deionized water, normal saline, phosphate-buffered saline, tris-buffered saline, sodium acetate buffer, sodium citrate buffer, or the like or combinations thereof.

In various embodiments, the mixing step (c-ii) is performed at physiological pH range of from about 7.00 to about 7.80, from about 7.05 to about 7.75, from about 7.10 to about 7.70, from about 7.15 to about 7.65, from about 7.20 to about 7.60, from about 7.25 to about 7.55, from about 7.30 to about 7.50, from about 7.35 to about 7.45, about 7.36, about 7.37, about 7.38, about 7.39, about 7.40, about 7.41, about 7.42, about 7.43, about 7.44, or about 7.45.

In various embodiments, the composition as disclosed herein comprises organic phase. In various embodiments, the aqueous composition comprises aqueous phase. In various embodiments, the mixing step (c-ii) comprises mixing the aqueous composition with the composition as described herein at a volume ratio of the aqueous phase to organic phase from about 10:1 to about 1:1. For example, the aqueous phase may be mixed with the organic phase at a volume ratio of from about 10:1 to about 1:1, at about 9:1, at about 8:1, at about 7:1, at about 6:1, at about 5:1, at about 4:1, at about 3:1, or at about 2:1.

In various embodiments, the step (c-ii) of mixing the aqueous composition with the composition comprises micro-mixing, e.g., microfluidic mixing using a microfluidic device. The micro-mixing may be performed via passive mixing using passive micromixers such as T-shaped or Y-shaped microfluidic mixers parallel lamination, sequential, focusing enhanced mixers or droplet micromixers. The micro-mixing may also be performed via active mixing using external forces such as pressure field, electrokinetic, dielectrophoretic, electrowetting, magneto-hydrodynamic or ultrasound. Advantageously, as microfluidic mixing comprises mixing the two compositions (i.e. aqueous composition and composition disclosed herein) in a controlled manner and/or with a specified/fixed/controlled mixing ratio, the interaction between the two compositions (e.g., between anionic lipid and cationic therapeutic and/or cationic prophylactic agent) is regulated, thereby producing nanoparticles with a smaller particle size and/or with a narrow size distribution or homogeneity (e.g., smaller PDI).

In various embodiments, there is provided anionic lipid nanoparticles for encapsulation and delivery of cationic macromolecules (e.g., cationic antimicrobial macromolecules).

In various embodiments, there is also provided a carrier, nanocarrier or delivery system/vehicle comprising the composition/compound/nanoparticles as disclosed herein.

In various embodiments, there is also provided a carrier, a nanocarrier, a delivery system/vehicle, a compound or ionized form thereof, a nanoparticle composition, nanoparticles (or anionic lipid nanoparticles) disclosed herein for use in medicine (e.g., for the treatment or prophylaxis of one or more of the diseases, disorders or conditions mentioned herein).

In various embodiments, there is also provided a carrier, a nanocarrier, a delivery system/vehicle, a compound or ionized form thereof, a nanoparticle composition, nanoparticles (or anionic lipid nanoparticles) disclosed herein for use in the treatment or prophylaxis of a disease, disorder or condition, the use of said carrier, a nanocarrier, a delivery system/vehicle, a compound or ionized form thereof, a nanoparticle composition, nanoparticles (or anionic lipid nanoparticles) in the manufacture of a medicament for the treatment or prophylaxis of a disease, disorder or condition and/or a method of treatment or prophylaxis of a disease, disorder or condition, comprising a step of administering (e.g. in a therapeutically effective amount of) said carrier, a nanocarrier, a delivery system/vehicle, a compound or ionized form thereof, a nanoparticle composition, nanoparticles (or lipid nanoparticles) to a subject (e.g., vertebrate such as a human or a large veterinary mammal (e.g., horses, cattle, deer, sheep, llamas, goats, pigs) in need thereof.

In various embodiments, the disease, disorder or condition is selected from infectious/contagious diseases, viral infections (i.e. diseases caused by virus), bacterial infections (i.e. diseases caused by bacteria), fungal infections (i.e. diseases caused by fungi), parasitic infections (i.e. diseases caused by parasite), respiratory diseases, or the like or combinations thereof. In various embodiments, the disease, disorder or condition is mediated by drug resistant bacteria, multidrug-resistant bacteria (MRD) and/or extremely drug-resistant (XDR) bacteria. In various embodiments, the disease, disorder or condition is mediated by Gram-negative bacteria. The disease, disorder or condition may be mediated by drug resistant bacteria and/or multidrug-resistant bacteria such as Gram-negative bacteria selected from Acinetobacter baumannii, Escherichia coli, Pseudomonas aeruginosa, or the like or combinations thereof.

In various embodiments, there is also provided a carrier, a nanocarrier, a delivery system/vehicle, a compound or ionized form thereof, a nanoparticle composition, nanoparticles (or lipid nanoparticles) disclosed herein for use in encapsulating and/or delivering a cationic therapeutic, and/or cationic prophylactic agent to a subject, cell, cytosol, tissue or organ (e.g., a mammalian cell, cytosol, tissue or organ), the use of said carrier, a nanocarrier, a delivery system/vehicle, a compound or ionized form thereof, a nanoparticle composition, nanoparticles (or lipid nanoparticles) in the manufacture of a medicament for encapsulating and/or delivering a cationic therapeutic, and/or cationic prophylactic agent to a subject, cell, cytosol, tissue or organ (e.g., a mammalian cell, cytosol, tissue or organ), and/or a method of delivering a cationic therapeutic, and/or cationic prophylactic agent to a subject, cell, cytosol, tissue or organ (e.g., a mammalian cell, cytosol, tissue or organ), comprising a step of administering (e.g. in a therapeutically effective amount of) said carrier, a nanocarrier, a delivery system/vehicle, a compound or ionized form thereof, a nanoparticle composition, nanoparticles (or lipid nanoparticles) to a subject (e.g., vertebrate such as a human or a large veterinary mammal (e.g., horses, cattle, deer, sheep, llamas, goats, pigs)) in need thereof.

In various embodiments, there is also provided a carrier, a nanocarrier, a delivery system/vehicle, a compound or ionized form thereof, a nanoparticle composition, nanoparticles (or lipid nanoparticles) disclosed herein for use in treating or preventing a bacterial infection in a subject (e.g., vertebrate such as a human or a large veterinary mammal (e.g., horses, cattle, deer, sheep, llamas, goats, pigs)), the use of said carrier, a nanocarrier, a delivery system/vehicle, a compound or ionized form thereof, a nanoparticle composition, nanoparticles (or lipid nanoparticles) in the manufacture of a medicament for treating or preventing a bacterial infection in a subject, and/or a method of treating or preventing a bacterial infection in a subject, comprising a step of administering (e.g. in a therapeutically effective amount of) said carrier, a nanocarrier, a delivery system/vehicle, a compound or ionized form thereof, a nanoparticle composition, nanoparticles (or lipid nanoparticles) to a subject in need thereof. The carrier, nanocarrier, delivery system/vehicle, compound or ionized form thereof, nanoparticle composition, nanoparticles may be delivered to a subject in the form of or as a component of an anti-microbial agent, anti-bacterial agent, anti-fungal agent, anti-viral agent, anti-parasitic agent or an antibiotic agent.

In various embodiments, the carrier, nanocarrier, delivery system/vehicle, compound or ionized form thereof, nanoparticle composition, nanoparticles prepared from embodiments of the method disclosed herein comprises one or more of the following characteristics or properties: broad applicability (e.g., can be used to encapsulate and/or deliver a wide range of cationic therapeutic, and/or cationic prophylactic reagents), nanosized, substantially neutral surface charge, high encapsulation efficiency (e.g., >80%), high antibacterial activity, high antimicrobial activity, high stability, low toxicity (e.g., low cytotoxicity, cytotoxicity, neurotoxicity, nephrotoxicity or the like), low production/synthesis cost, therefore making them suitable for in vivo applications that require efficient cellular uptake.

In various embodiments, the compound comprises one or more carboxylic acid groups. Advantageously, in various embodiments, the carboxylic acid acts as a binding group to condense cationic molecules/cargoes (e.g., antibiotics) through electrostatic interaction into lipid nanoparticles (LNPs).

### BRIEF DESCRIPTION OF FIGURES

FIG. 1 shows 1H NMR spectrum of Oligoglutamate₅-Succinic-Lipid designed in accordance with various embodiments disclosed herein in DMSO-d6.

FIG. 2 shows liquid chromatography-mass spectrometry (LC-MS) spectra of the final synthesized oligoglutamate₅-Succinic-Lipid designed in accordance with various embodiments disclosed herein. Positive MS mode is shown in the top figure while negative MS mode is shown in the bottom figure.

FIG. 3 shows a calibration plot of various free polymyxin B standards with the corresponding peak areas at retention time ~5.30 min.

FIG. 4 shows the viability of SH-SY5Y cells after 24 h of incubation with free polymyxin B or polymyxin B-loaded LNPs designed in accordance with various embodiments disclosed herein at 300 μg mL⁻¹. Data represent means±SD (n=18, biologically independent samples). The asterisk indicates that differences between the groups are statistically significant using a two-tailed unpaired Welch's t-test. (****: p<0.001). SPSS statistical analyses also confirmed the same results.

FIG. 5 shows the viability data of SH-SY5Y cells comparing free polymyxin B and polymyxin B-loaded LNPs designed in accordance with various embodiments disclosed herein at 300 μg mL⁻¹ using a normality Q-Q plot. Incubation time: 24 h.

### EXAMPLES

Example embodiments of the disclosure will be better understood and readily apparent to one of ordinary skill in the art from the following examples, tables and if applicable, in conjunction with the figures. It should be appreciated that other modifications related to structural, and/or chemical changes may be made without deviating from the scope of the disclosure.

The following examples describe the development of an anionic lipid nanoparticle formulation comprising an anionic Oligo-glutamates-Succinic-Lipid conjugate that effectively encapsulates and delivers a cationic therapeutic and/or cationic prophylactic agent (e.g., polymyxin B) with comparable MIC/MBC and reduced cytotoxicity. Advantageously, the present application has shown that the formulations designed in accordance with various embodiments disclosed herein comprising an anionic oligo (glutamate) 5 lipid (i.e., Oligo-glutamates-Succinic-Lipid conjugate) with cationic PB form lipid nanoparticles (LNPs) with colloidal stability, lower neurotoxicity, and with comparable efficacy against Gram-negative Escherichia coli and Pseudomonas aeruginosa. Notably, after 7 days of storage at 4° C., the hydrodynamic size and neutral surface charge of the LNPs remained unchanged in both 0.9% saline (pH 7.4) and 10% FBS-containing saline (0.9%, pH 7.4) and showed no significant aggregation of LNPs. Even more advantageously, the encapsulation efficiency of the formulations designed in accordance with various embodiments disclosed herein with a varying amount of PEG-lipid conjugate is as high as 29%-62%. Unlike free polymyxin B which showed relatively pronounced cell toxicity against an SH-SY5Y neurotoxicity cell model than the LNPs encapsulating PB at a concentration of 300 μg mL⁻¹, the LNPs encapsulating PB designed in accordance with various embodiments disclosed herein showed comparable MIC values to free PB, thereby representing a good candidate for the treatment of bacterial infections, e.g., Gram-negative bacterial infection.

### Example 1: Materials and Methods

### 1.1. Materials

All chemicals were purchased from Sigma-Aldrich, Alfa Chemistry, GL Biochem (Shanghai), and Tokyo Chemical Industry (Singapore) and used as received unless specified. Polymyxin B was purchased from Merck. Solvents purchased from VWR, Fisher Scientific, Fulltime, or J. T. Baker, were of high-performance liquid chromatography or analytical grade and used as received. Anionic oligopeptide-lipid conjugate was synthesized via a solid-phase strategy using a protected anionic amino acid, 2-Chlorotrityl Chloride (CTC) resin (loading factor: 1.190 mmol/g), HBTU, and HOBt were purchased from GL Biochem Ltd. (Shanghai). Deuterated solvents were purchased from Cambridge Isotope Laboratories (USA). The lipids 1,2-distearoyl-sn-glycerol-3-phosphocholine (DSPC), cholesterol, and ALC-0159, which were used to make LNPs with the anionic oligopeptide-lipid conjugate and polymyxin B, were purchased from MedChemExpress (Monmouth Junction, NJ, USA). Polymyxin B sulfate was purchased from EMD Chemicals (San Diego, CA, USA). Fetal bovine serum (FBS) was purchased from (Corning, USA), and 0.9% w/v saline (pH 7.4) from (B Braun, Singapore). Immortalized human embryonic kidney cell line (HEK293) and thrice-subcloned cell line derived from the SK-N-SH neuroblastoma cell line (SH-SY5Y) were obtained from ATCC (U.S.A). AlamarBlue Cell Viability reagents were purchased from ThermoFisher Scientific, Singapore. Phosphate-buffered saline (PBS, 10×) was purchased from 1st BASE and diluted to 1×PBS before use. Cation-adjusted Mueller-Hinton broth (MHB) powder was bought from BD Diagnostics and used to prepare the microbial broth according to the manufacturer's instructions. LB Agar Powder, Miller, was bought from Bio Basic and used to prepare LB agar plates according to the manufacturer's instructions. Escherichia coli (ATCC No. 25922) and Pseudomonas aeruginosa (ATCC No. 9027) were purchased from ATCC (U.S.A.) and reconstituted according to the suggested protocols outlined in the methods section.

### 1.2. Solid-Phase Synthesis of Anionic Glutmate₅-Succinic-Lipid Conjugate

The synthesis of anionic Glutmate₅-Succinic-Lipid is depicted in Scheme 1. The 2-Chlorotrityl Chloride Resin (2.00 mmol, 1680 mg) was first stirred and swelled in DMF (10 mL) at 25° C. for 0.5 h. Next, a pre-mixed solution of Fmoc-L-Glu (tBu) (2.50 mmol, 850.36 mg) and N,N-diisopropylethylamine (DIPEA, 2.50 mmol, 440 μL) in DMF (50 mL) was added to the resin. The mixture was stirred at 24° C. for 0.5 h. Unreacted reagents were removed by filtration, and the resin was washed with MeOH (3×30 mL) and CH₂Cl₂ (3×30 mL) followed by DMF (3×30 mL). Next, Fmoc removal was performed by adding 20% piperidine in DMF (v/v, 50 mL) to the resin. The mixture was stirred at 24° C. for 0.15 h. Unreacted reagents were removed by filtration, and the resin was washed with MeOH (3×30 mL) and CH₂Cl₂ (3×30 mL) followed by DMF (3×30 mL). A pre-mixed solution of Fmoc-L-Glu(tBu)-OH (2.50 mmol, 1.06 g), HBTU (2.50 mmol, 0.95 g), HOBt (2.50 mmol, 0.34 g) and N,N-diisopropylethylamine (DIPEA, 2.50 mmol, 440 L) were dissolved in DMF (v/v, 50 mL) and added to the resin. The mixture was stirred at 80° C. for 0.5 h. Unreacted reagents were removed by filtration, and the resin was washed with MeOH (3×30 mL) and CH₂Cl₂ (3×30 mL) followed by DMF (3×30 mL). The Fmoc-removal and Fmoc-L-Glu(tBu)-OH coupling steps were repeated three more times to obtain Resin-Glu(tBu) 5-Fmoc. Finally, Fmoc removal was performed by adding 20% piperidine in DMF (v/v, 50 mL) to the resin. The mixture was stirred at 24° C. for 0.15 h. Unreacted reagents were removed by filtration, and the resin was washed with MeOH (3×30 mL) and CH₂Cl₂ (3×30 mL) followed by DMF (3×30 mL). Next, a pre-mixed solution of succinic anhydride (5.00 mmol, 500.35 mg) and N,N-diisopropylethylamine (DIPEA, 5.00 mmol, 880 μL) in DMF (50 mL) was added to the resin and stirred for 2 h. Unreacted reagents were removed by filtration, and the resin was washed with MeOH (3×30 mL) and CH₂Cl₂ (3×30 mL) followed by DMF (3×30 mL). A pre-mixed solution of ditetradecylamine (2.50 mmol, 1.062 g), HBTU (2.50 mmol, 0.95 g), HOBt (2.50 mmol, 0.34 g) and N,N-diisopropylethylamine (DIPEA, 2.50 mmol, 440 μL) were dissolved in DMF (v/v, 50 mL) and added to the resin. The mixture was stirred at 80° C. for 0.5 h. Unreacted reagents were removed by filtration, and the resin was washed with DMF (3×30 mL) and MeOH (3×30 mL) followed by CH₂Cl₂ (3×30 mL). The targeted Glutamic acid₅-Succinic-Lipid was cleaved from the resin using 95% TFA in CH₂Cl2 (v/v, 20 mL) at 25° C. over 0.5 h. A gentle stream of N₂ gas removed excess TFA to give a brown oil and further dissolved in MeOH (5 mL) and crystallized by cold diethyl ether (45 mL). The crystallization purification step was repeated 3 times and lyophilized to give an off-white powder.

LC-MS analyses of the intermediate products were measured on an LCMS-2020 spectrophotometer (Shimadzu, Singapore). 1H NMR spectra of the anionic Oligo-glutamates-Succinic-Lipid conjugate was recorded on a Bruker AV 400M NMR spectrometer in DMSO-d6 (¹H at 400 MHz). 1H NMR spectra data are reported as follows: chemical shift δ (ppm) referenced to either DMSO-d6 (2.50 ppm), coupling constant J (Hz), and integration.

An exemplary scheme for the synthesis of a compound comprising a structure represented by general formula (1) or ionized forms thereof in accordance with various embodiments disclosed herein is provided in Scheme 1.

### Solid-Phase Synthesis of Glutamate₅-Succinic-C14Lipid

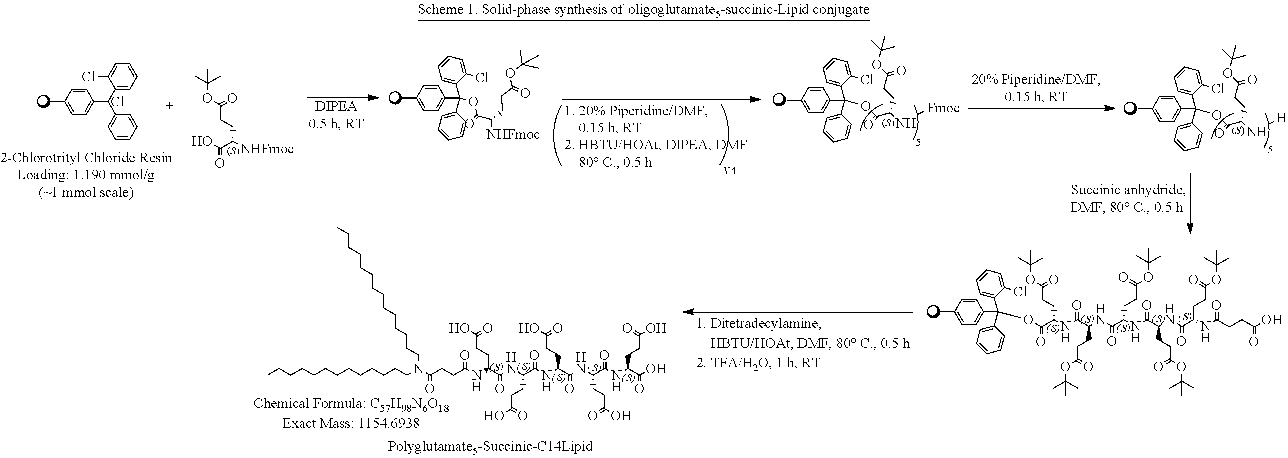

### 1.3. Polymyxin B-Loaded LNPs Fabrication

The polymyxin B-loaded LNPs were formulated in a controlled mixing procedure between cationic polymyxin B-containing aqueous phase and lipid mixture in ethanol solvent containing cholesterol, DSPC, PEG Lipid ALC-0159 and Oligoglutamate₅-Succinic-Lipid using NanoAssemblr Spark or Benchtop (Precision NanoSystems). The oligoglutamate₅-Succinic-C14Lipid was pre-dissolved in ethanol/DMSO solvent (9:1 v/v) at a 5 mg/mL concentration for subsequent use. The overall flow rate was maintained at 12 mL per min, a 3:1 volume ratio of aqueous to organic phase for formulating the LNPs using a microfluidic device (NanoAssemblr® Ignite™, Precision Nanosystem, Vancouver, CA, USA). A charge ratio of polymyxin B (+ve): Oligoglutamate₅-Succinic-C14Lipid (−ve) (4:1) was maintained throughout the study. The formulated LNPs contains polymyxin B (sulfate form, MW=1301, Free form, MW=1202), helper lipid 1,2-distearoyl-sn-glycero-3-phosphorylcholine (DSPC), Cholesterol, Oligoglutamate₅-Succinic-Lipid (MW=1154; structure illustrated in Scheme 1), and PEG-lipid (ALC-0159) in two different varied formulations at a molar ratio of 38.4:9.4:42.7:8.0:1.5 (1^{st} Formulation) and 37.2:9.4:42.7:7.7:3.0 (2^{nd} Formulation), respectively. The removal of surplus ethanol, DMSO, and unencapsulated polymyxin B was achieved by diluting the resultant LNPs in 0.9% saline (pH 7.4) and further concentrated via centrifugal ultrafiltration using Amicon Ultra centrifugal filters Units (MWCO: 30,000 Da; EMD Millipore).

### 1.4. LNPs Characterization and Stability Assessment by Dynamic Light Scattering and Zeta Potential (ζ) Measurements

The LNPs suspension was diluted by 50 times in either 0.9% saline (pH 7.4) or 10% FBS-containing saline (pH 7.4), and the particle size distribution and ζ potential were measured three times for each sample using a Zetasizer Nano-ZS90 (Malvern, UK). The stability study of LNPs was performed in saline (pH 7.4) and saline containing 10% FBS by measuring particle size at pre-determined time points for 7 days at 4° C. LNPs size (Z-average and polydispersity index (PDI) are reported as the mean±standard deviation (n=3 measurements).

For zeta potential measurements, 20 μL of each formulated LNPs was diluted 50× in saline (pH 7.4) and pipetted into DTA1070 zeta potential cuvettes (Malvern Panalytical, Malvern, UK) and measured on the Zetasizer Nano instrument. The zeta potential of LNPs is reported as the mean±standard deviation (n=3 measurements). The same procedure was performed to measure the zeta potential of the LNPs in saline containing 10% FBS.

### 1.5. Determination of Encapsulation Efficiency (EE) and Polymyxin B Content in the LNPs by HPLC

Shimadzu ultra high-speed LC/MS Prominence UFLC+LCMS-2020 high-performance liquid chromatography system with a gradient pump was used for polymyxin B analyses. The liquid chromatography was performed on a Symmetry C18 column (250 mm×4.6 mm, 5 μm, 100 Å) with water containing 0.1% formic acid (A) and acetonitrile containing 0.1% formic acid (B) as the mobile phase. The flow rate was 1 mL/min, and the detection wavelength was 215 nm. The oven temperature was set at 24° C. Polymyxin B stock solution was prepared in 0.9% saline solution (2.00 mg/mL) and sequentially diluted with 0.9% saline solution to 1.75, 1.50, 1.25, 1.00, 0.75, 0.50, 0.25 mg/mL to establish the standard curve. Each standard concentration was repeated three times. Formulated LNPs were treated with 0.5% w/v Triton X-100 (Sigma-Aldrich, Cat #T8787) and incubated at 37° C. for 0.5 h to disrupt the LNP structure to release polymyxin B and analyzed through LC-MS. The peak area of the sample further calculated the content of polymyxin B at retention time (~5.30 min). Encapsulation efficiency was then reported as a calculated value,

$$EE ( \% ) = ( \frac{Rx - RTE}{R x} ) \times 100 \% ,$$

where R^{x} is the polymyxin B content in the Triton X-100 mixture, and RTE is the polymyxin B content in the 0.9% saline solution.

### 1.6. In Vitro Nephrotoxicity and Neurotoxicity Assessments Using HEK 293 and SH-SY5Y Cell Lines, Respectively

HEK293 cells were grown in Dulbecco's Minimum Essential Medium (DMEM) supplemented with 10% FBS and 5% penicillin/streptomycin (Corning). SY-SY5Y cells were grown with a mixture of DMEM/Ham's F-12 with 2 mM L-glutamine (50:50 v/v) supplemented with 10% FBS and 5% penicillin/streptomycin. All cultures were grown in 37° C. incubators supplemented with 5% CO₂ and were cultured according to suppliers' instructions. For all screening, cells were plated in 96-well plates at 10,000 cells per well in 100 μL of the appropriate media outlined above for the different cells. The formulated LNPs containing polymyxin B and free polymyxin B were then added to the wells at the desired polymyxin B concentration (e.g., 300, 150, 75, 37.5, 18.8, 9.4 μg mL⁻¹). The polymyxin B concentration of formulated LNPs was determined using the calibration plot of polymyxin B standards.

### 1.7. Antibacterial Activity In Vitro

The MICs (minimum inhibitory concentrations) of the formulated LNPs containing polymyxin B and free polymyxin B against Gram-negative bacteria were determined using a broth microdilution method. Briefly, the bacteria were incubated overnight in MHB, and the bacteria suspension was diluted with MHB to achieve a concentration of 108 colony-forming units (CFU)/mL (O.D. 600=0.100). This was followed by a 1000-fold dilution in MHB. The bacterial suspension was then seeded into a 96-well plate (80 μL per well) and mixed with 20 μL of MHB containing different polymyxin B-loaded LNPs or free polymyxin B concentrations. The 96-well plate was incubated for 24 h at 37° C. (bacteria). The MIC was determined as the lowest concentration of the polymyxin B-loaded or free polymyxin B, at which no turbidity changes were measured using a Spark 10M multimode microplate reader (TECAN, Switzerland). The MIC experiments were performed with four independent replicates. The MBCs (minimum bactericidal concentrations) were evaluated at the end of the MIC experiment.

The microbial suspension was then spread on LB agar plates, and the plates were incubated at 37° C. for 24 h to determine CFU on each agar plate. The experiments were performed in replicates of five. The MBC experiment was conducted with three independent replicates.

### 1.8. Hemolysis Assay

The toxicity of the polymyxin B-loaded LNPs and free polymyxin B against mammalian erythrocytes was tested using fresh rat red blood cells (rRBCs). Briefly, rRBCs were diluted 25-fold in PBS to achieve 4% v/v of blood content. The LNPs or polymyxin B was dissolved in PBS at concentrations ranging from 0 to 4000 μg mL⁻¹ by serial dilutions. Equal volumes of the solution (100 μL) were then mixed with the diluted blood suspension (100 μL). The mixtures were then incubated at 37° C. for 1 h to allow interactions between rRBC and the LNPs or polymyxin B to occur. After that, the mixture was subjected to centrifugation (1000 g for 5 min, 4° C.), and 100 μL aliquots of the supernatant was pipetted into a 96-well microplate. The hemoglobin release was measured spectrophotometrically by measuring the absorbance of the samples at 576 nm using the microplate reader. Two control groups were employed for this assay: untreated rRBC suspension (negative control), and rRBC suspension treated with 0.1% Triton-X (positive control). Each assay was performed in 4 replicates. The percentage of hemolysis was defined as follows: Hemolysis (%)=[(OD₅₇₆ nm of the treated sample−OD₅₇₆ nm of the negative control)/(OD₅₇₆ nm of positive control−OD₅₇₆ nm of negative control)]×100%.

### 1.9. Statistical Analysis

Statistical analyses for various data were performed using GraphPad Prism v.9.0.0 (GraphPad Software) and SPSS Statistics. The Statistical significance analyses were determined by a Two-tailed unpaired t-test with Welch's correction. Q-Q plots were also used to determine whether the sampled data follows a Gaussian (normal) distribution and the Welch's correct t-test statistical analysis.

### Example 2: Results and Discussion

### 2.1. Synthesis and Characterization of Oligoglutamate₅-Succinic-Lipid

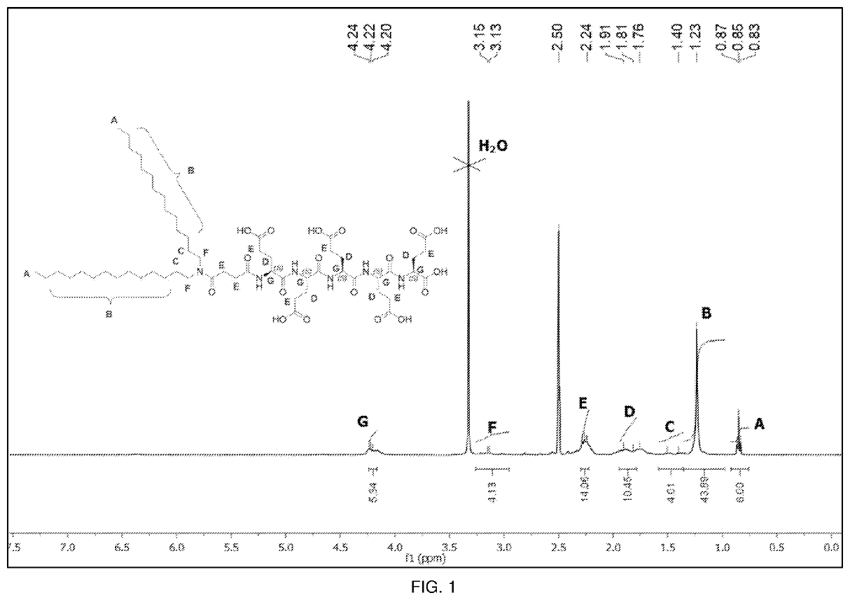

The solid-phase synthesis of oligoglutamate₅-Succinic-Lipid was performed. The target compound was obtained and lyophilized overnight in vacuo to give an off-white powder (208 mg, yield=9%). ¹H NMR spectroscopy was performed on the target compound and was successfully characterized (FIG. 1). 1H NMR (400 MHz, DMSO) δ 4.24-4.17 (m, 5H), 3.14 (d, J=6.2 Hz, 4H), 2.26 (dd, J=12.6, 5.8 Hz, 14H), 1.86 (d, J=36.8 Hz, 10H), 1.45 (d, J=42.2 Hz, 4H), 1.23 (s, 44H), 0.85 (t, J=6.8 Hz, 6H).

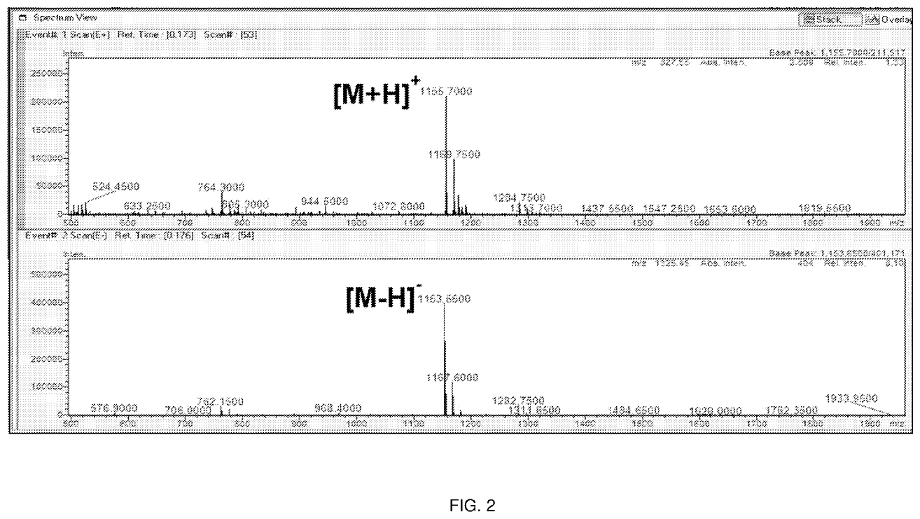

LC-MS purity analysis on Oligoglutamate₅-Succinic-Lipid was performed, giving an overall ~98% purity (FIG. 2). Chemical Formula: C₅₇H₉₈N₆O₁₈, Exact Mass: 1154.6938 Found: [M+H]⁺=1155.7000, [M−H]⁻=1153.6500.

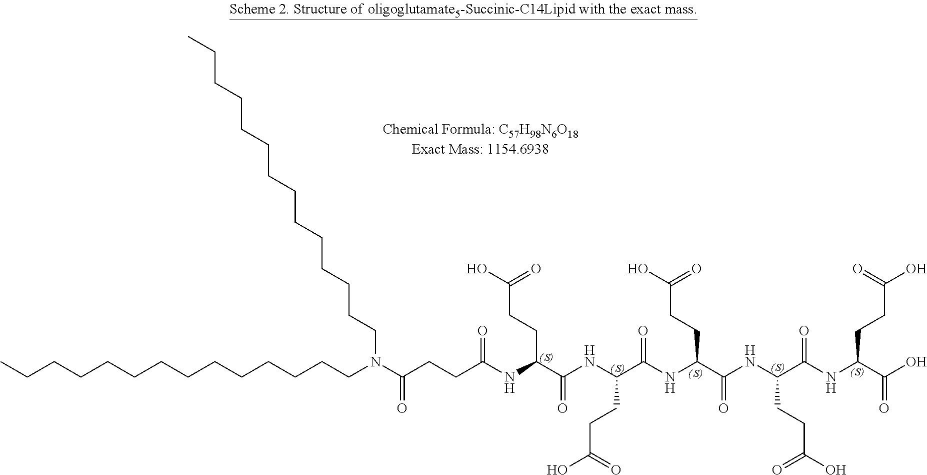

### 2.2. Characterization of the Polymyxin B-Loaded LNPs

The LNPs containing polymyxin B in two different formulations were synthesized using a microfluidic device, and the preparation conditions are outlined in Tables 1 and 2.

The LNPs formed from the 1^{st} and 2^{nd} formulations had an average size of 74 and 85 nm, respectively. The polydispersity index also had a narrow size distribution (PDI: 0.15-0.18), and the surface zeta potential was close to neutral (Tables 3 and 4), which is ideal for in vivo applications.

TABLE 1
Preparation parameters of the LNPs - 1^{st} Formulation with 1.5% ALC-0159

| | Molecular | Stock | Amount | | Molar |
|---|---|---|---|---|---|
| | Weight | Concentration | Added | Volume | Percentage |
| Lipid | (mg/mmoL) | (mg/mL) | (mg) | (μL) | (%) |
| Polymyxin B sulfate | 1385.61 | 2 | 1.943 | 971.7 | 38.4 |
| Nuclease-Free Water | — | — | — | 528.3 | — |
| DSPC | 790.15 | 10 | 0.289 | 28.9 | 9.4 |
| Cholesterol | 386.65 | 20 | 0.642 | 32.1 | 42.7 |
| Polyglutamate₅-Succinic-Lipid | 1154.69 | 5 | 0.359 | 71.8 | 8.0 |
| Ethanol | — | — | — | 319.6 | — |
| ALC-0159 | 2450.00 | 3 | 0.143 | 47.6 | 1.5 |

The net volume for the aqueous (1.5 mL) and organic phases (0.5 mL) was 3 mL.
Charge ratio (+ve/−ve) = 4:1

TABLE 2
Preparation parameters of the LNPs - 2^{nd} formulation with 3.0% ALC-0159

| | Molecular | Stock | Amount | | Molar |
|---|---|---|---|---|---|
| | Weight | Concentration | Added | Volume | Percentage |
| Lipid | (mg/mmoL) | (mg/mL) | (mg) | (μL) | (%) |
| Polymyxin B sulfate | 1385.61 | 2 | 1.880 | 940.2 | 37.2 |
| Nuclease-Free Water | — | — | — | 559.8 | — |
| DSPC | 790.15 | 10 | 0.289 | 28.9 | 9.4 |
| Cholesterol | 386.65 | 20 | 0.642 | 32.1 | 42.7 |
| Polyglutamate₅-Succinic-Lipid | 1154.69 | 5 | 0.347 | 69.5 | 7.7 |
| Ethanol | — | — | — | 274.3 | — |
| ALC-0159 | 2450.00 | 3 | 0.286 | 95.3 | 3.0 |

The net volume for the aqueous (1.5 mL) and organic phases (0.5 mL) was 3 mL.
Charge ratio (+ve/−ve) = 4:1

TABLE 3
Particle size, size distribution, and zeta potential of the LNPs using the 1^{st} formulation
parameters across Days 1, 2, 3, and 7 at 4° C.

| 1^{st} | | | | | | |
|---|---|---|---|---|---|---|
| Formulation | DLS Size | | | | Zeta Potential (mV) | |
| Polymyxin | Saline 0.9% | PDI in Saline | 10% FBS | PDI in 10% | Saline 0.9% | 10% FBS Saline |
| B- | pH 7.4 | 0.9% | Saline 0.9% | FBS Saline | PH 7.4 | 0.9% pH 7.4 |
| LNPs | Size (nm) | pH 7.4 | Size (nm) | 0.9% pH 7.4 | ζ (mV) | ζ (mV) |
| Day 1 | 73.50 ± 0.16 | 0.15 ± 0.03 | 78.80 ± 0.13 | 0.15 ± 0.03 | 3.153 ± 1.61 | −9.660 ± 2.41 |
| Day 2 | 75.40 ± 0.21 | 0.18 ± 0.08 | 76.65 ± 1.36 | 0.25 ± 0.04 | −6.260 ± 1.09 | −7.377 ± 1.94 |
| Day 3 | 76.14 ± 0.67 | 0.16 ± 0.01 | 74.26 ± 1.59 | 0.38 ± 0.08 | −9.612 ± 1.69 | −12.12 ± 2.16 |
| Day 7 | 76.65 ± 1.36 | 0.25 ± 0.04 | 70.45 ± 1.98 | 0.45 ± 0.02 | −7.38 ± 1.94 | −6.26 ± 1.09 |

TABLE 4
Particle size, size distribution, and zeta potential of the LNPs using the 2^{nd} formulation
parameters across Days 1, 2, 3, and 7 at 4° C.

| 2^{nd} | | | | | | |
|---|---|---|---|---|---|---|
| Formulation | DLS Size | | | | Zeta Potential (mV) | |
| Polymyxin | Saline 0.9% | PDI in Saline | 10% FBS | PDI in 10% | Saline 0.9% | 10% FBS Saline |
| B- | pH 7.4 | 0.9% | Saline 0.9% | FBS Saline | PH 7.4 | 0.9% pH 7.4 |
| LNPs | Size (nm) | pH 7.4 | Size (nm) | 0.9% pH 7.4 | ζ (mV) | ζ (mV) |
| Day 1 | 89.38 ± 2.4 | 0.15 ± 0.02 | 78.06 ± 6.98 | 0.52 ± 0.01 | 3.153 ± 1.61 | −6.150 ± 3.74 |
| Day 2 | 84.24 ± 2.8 | 0.15 ± 0.01 | 75.04 ± 10.96 | 0.54 ± 0.06 | −8.225 ± 3.31 | −9.300 ± 1.82 |
| Day 3 | 84.86 ± 1.2 | 0.15 ± 0.02 | 72.90 ± 15.24 | 0.57 ± 0.15 | −9.300 ± 1.82 | −11.770 ± 3.69 |
| Day 7 | 84.84 ± 1.4 | 0.15 ± 0.02 | 75.16 ± 37.36 | 0.59 ± 0.11 | −5.019 ± 1.94 | −8.137 ± 0.67 |

### 2.3. Colloidal Stability of the Polymyxin B-Loaded LNPs

After 7 days of storage at 4° C., the size of the LNPs made from both the 1^{st} and 2^{nd} formulation remained unchanged in both 0.9% saline and 10% FBS-containing saline (Tables 3 and 4). PDI of the LNPs made from 1^{st} formulation was comparable in both saline and 10% FBS-containing saline after 1 day of storage at 4° C. (PDI: 0.15, single peak except for FBS peak from the dynamic light scattering analysis), while that of the LNPs made from 2^{nd} formulation was larger in 10% FBS-containing saline than saline even after 1 day of storage at 4° C. (PDI: 0.52, two peaks except for FBS peak from the dynamic light scattering analysis), suggesting aggregation. This result demonstrated that 1^{st} formulation was more stable than 2^{nd} formulation. The relatively neutral surface charges of the LNPs over 7 days made from both formulations were close to neutral within ±10 mV. Notably, the superior colloidal stability of LNPs especially made from 1^{st} formulation under serum conditions, could bestow enhanced bioavailability in vivo, which is advantageous as therapeutics against Gram-negative bacterial infections.

### 2.4. Encapsulation Efficiency of Polymyxin B in LNPs

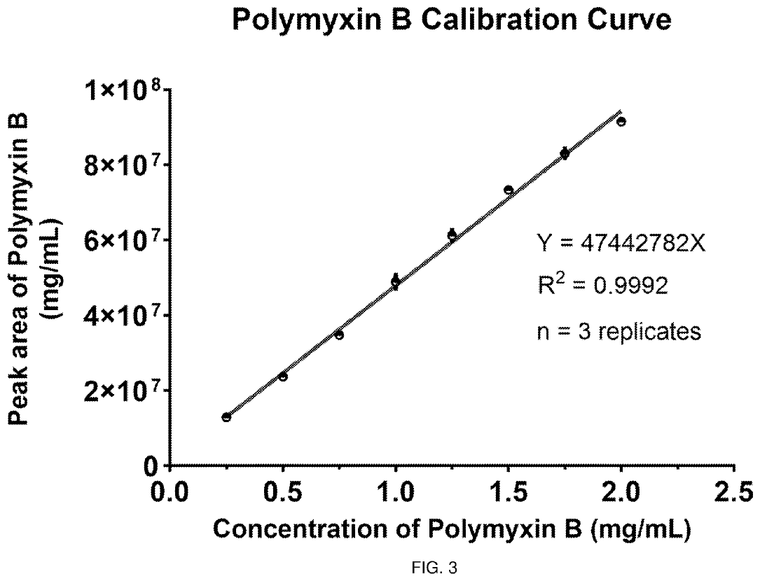

Reverse-phase HPLC was used to determine the encapsulation efficiency of polymyxin B in the LNPs. First, the free polymyxin B calibration curve of various concentration ranges (i.e., 0.25 to 2.00 mg mL⁻¹) was plotted using the peak areas under the curve at a retention time 5.30 min (Table 5). Mass spectrometry was used to confirm the identity of the peak that belongs to polymyxin B. The calibration plot gave the following equation Y=47442782X, where Y is the peak area under the curve around retention time 5.30 min and X is the concentration of polymyxin B. The coefficient of determination R² was 0.9992, showing good model prediction (FIG. 3). The individual peak areas of polymyxin B component were integrated at a retention time of 5.30 min with 0.5% triton (v/v) treated and without triton treatment in the final formulated LNPs. The encapsulated efficiency was calculated and tabulated, giving 29% and 62% for the 1st and 2nd formulations, respectively.

### 2.5. Biocompatibility of Polymyxin B-Loaded LNPs In Vitro

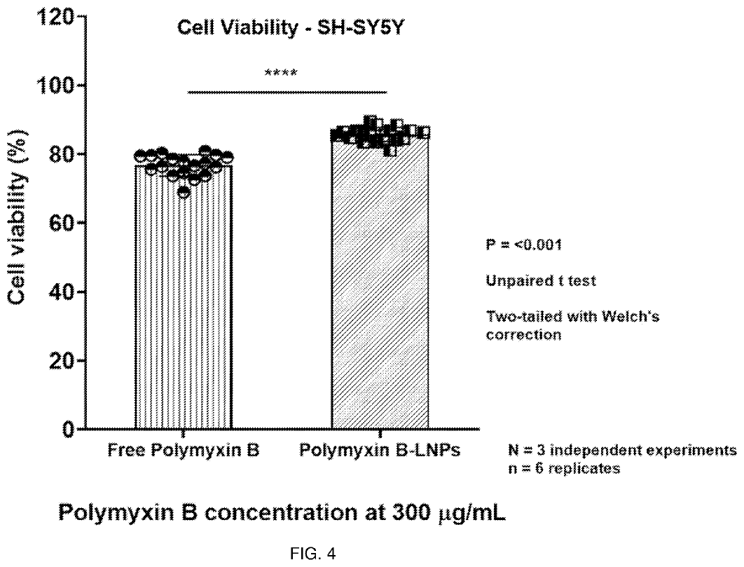

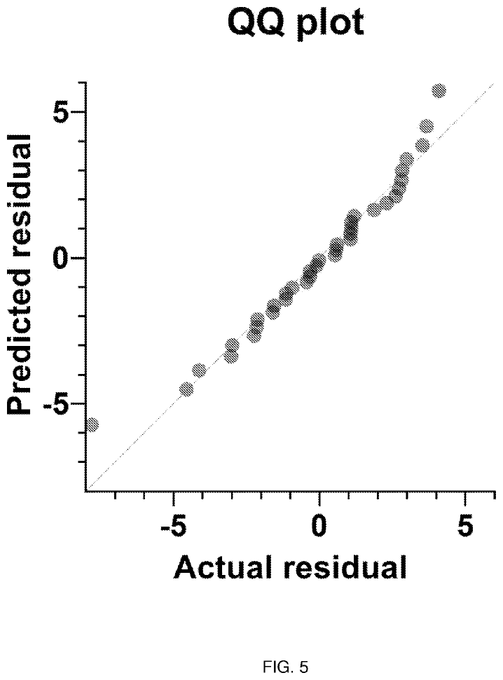

Both nephrotoxicity and neurotoxicity of polymyxin B have plagued their widespread use in clinics. Still, they remain essential for the use against Gram-negative multidrug-resistant (MDR) and extremely drug-resistant (XDR) infections. The cytotoxicity of polymyxin B-loaded LNPs from the 1^{st} formulation and free polymyxin B was thus evaluated at a high concentration of 300 μg mL⁻¹ of polymyxin B using SH-SY5Y cells as the neurotoxicity model. Notably, the viability of SH-SY5Y cells showed good tolerability for polymyxin B-loaded LNPs (~86%) but not free PB (~76%) after 24 h incubation. Statistical Two-tailed Welch's t-test at a 95% confidence level was calculated, indicating a statistically significant between the two groups with a P value=<0.001 (FIG. 4). The normality test Q-Q plot also confirmed that the data followed a Gaussian distribution (FIG. 5).

TABLE 5
Tabulated results of various free polymyxin B concentration standards
with the corresponding peak areas at retention time ~5.30 min.

| | Sample (Polymyxin B) | | | | Mean Area & |
|---|---|---|---|---|---|
| S/No | mg/mL | Peak Area 1 | Peak Area 2 | Peak Area 3 | Standard Deviation |
| 1 | 0.25 | 12737519 | 12884749 | 13092930 | 12884749 ± 13092930 |
| 2 | 0.50 | 24257286 | 23534205 | 23253307 | 23534205 ± 23253307 |
| 3 | 0.75 | 34456460 | 35108921 | 34840400 | 35108921 ± 34840400 |
| 4 | 1.00 | 50916179 | 47747278 | 47985117 | 47747278 ± 47985117 |
| 5 | 1.25 | 62778907 | 60382086 | 60394090 | 60382086 ± 60394090 |
| 6 | 1.50 | 74334505 | 72849637 | 72861641 | 72849637 ± 72861641 |
| 7 | 1.75 | 84420876 | 82335454 | 82347458 | 82335454 ± 82347458 |
| 8 | 2.00 | 92104554 | 91213765 | 91225769 | 91213765 ± 91225769 |

### 2.6. In Vitro Antimicrobial Activity of Polymyxin B-Loaded LNPs Against Gram-Negative Bacteria

The in vitro antimicrobial activity of the polymyxin B-loaded LNPs and free polymyxin B was ascertained against two Gram-negative bacteria strains (Escherichia coli, ATCC No. 25922) and Pseudomonas aeruginosa, ATCC No. 9027). The results are tabulated in Tables 6 and 7.

TABLE 6
MIC and MBC (μg mL⁻¹) of the polymyxin B-loaded LNPs from
the 2^{nd} formulation against bacteria. R = (MBC or MFC)/MIC.
R ≤ 4 signifies bactericidal activity.

| | Gram-negative | | | | | |
|---|---|---|---|---|---|---|
| | E. coli | | | P. aeruginosa | | |
| Samples | MIC | MBC | R | MIC | MBC | R |
| Polymyxin B (PB) | 1.25 | 5.0 | 4 | 1.25 | 2.50 | 2 |
| PB-loaded LNPs | 1.56 | 1.95 | 1.25 | 1.95 | 3.90 | 2 |

TABLE 7
MIC and MBC (μg mL⁻¹) of the polymyxin B-loaded LNPs
from the 1^{st} formulation against bacteria. R = (MBC)/MIC.
R ≤ 4 signifies good bactericidal activity.

| | Gram-negative | | | | | |
|---|---|---|---|---|---|---|
| | E. coli | | | P. aeruginosa | | |
| Samples | MIC | MBC | R | MIC | MBC | R |
| Polymyxin B (PB) | 5.0 | 5.0 | 1 | 5.0 | 5.0 | 1 |
| PB-loaded LNPs | 1.00 | 1.50 | 1.50 | 1.5 | 3.00 | 2 |
| (1^{st} formulation) | | | | | | |

The MIC and MBC values of polymyxin B-loaded LNPs made from 1^{st} formulation against both E. coli and P. aeruginosa were comparable to those of free polymyxin B, demonstrating that the encapsulation of polymyxin B in LNPs did not affect its antimicrobial activity significantly (Tables 6 and 7). In contrast, polymyxin B-loaded LNPs made from 2^{nd} formulation had higher MIC and MBC values than free polymyxin against both types of bacteria, demonstrating that the encapsulation of polymyxin B into the LNPs made from 2^{nd} formulation reduced the antimicrobial activity of polymyxin B. The MBC/MIC ratio (R) of each sampled antimicrobial agent could be utilized to ascertain their bactericidal potential, and studies have demonstrated that antimicrobial agents with R≤4 signify good bactericidal activity. Notably, the polymyxin B-loaded LNPs showed low R values of 1.25 and 2 against E. coli and P. aeruginosa, respectively, demonstrating the ability to represent as a good candidate for the treatment of Gram-negative bacterial infection.

### Example 3: Summary

The use of anionic lipids successfully encapsulated cationic polymyxin B into LNPs. These LNPs showed no significant aggregation in both normal saline and serum-containing medium, which is advantageous as therapeutics as it may provide improved bioavailability in vivo. Pronounced neurotoxicity was observed in free polymyxin B but not in the polymyxin B-loaded LNPs at high concentrations. Notably, the encapsulation of polymyxin B in LNPs did not affect its antimicrobial activity in Gram-negative Escherichia coli and Pseudomonas aeruginosa. Taken together, the polymyxin-loaded LNPs are a potentially promising therapeutic candidate for the treatment of Gram-negative bacterial infection. These anionic LNPs may also be used to deliver other cationic antimicrobial macromolecules, such as antimicrobial polymers and peptides.

The formulation of lipid nanoparticles from an anionic lipid and cationic PB increases the stability of PB, lowers nephrotoxicity and neurotoxicity, and exerts effective treatment against Gram-negative bacteria, thereby presenting an attractive therapeutic paradigm shift for clinical use.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A compound comprising a structure represented by general formula (1) or ionized forms thereof for preparing lipid nanoparticles encapsulating a cationic therapeutic and/or cationic prophylactic agent: 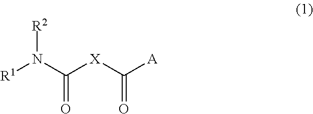
wherein
R¹ and R² are each independently a hydrophobic group;
X comprises an optionally substituted linear aliphatic, optionally substituted branched aliphatic and/or optionally substituted cyclic hydrocarbons; and
A comprises a peptide sequence of 1-5 anionic amino acids.

2. The compound of claim 1, wherein the structure is represented by general formula (1A): 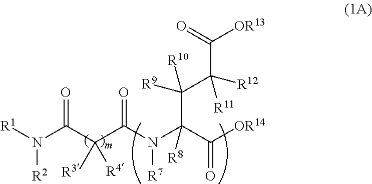
and wherein
R^{3′}, R^{4′}, R⁷ to R¹⁴ are each independently selected from the group consisting of H, optionally substituted alkyl, optionally substituted alkenyl, optionally substituted alkynyl, and combinations thereof;
m is 1, 2, 3, 4 or 5; and
n is 1, 2, 3, 4 or 5.

3. The compound of claim 1, wherein the hydrophobic group at R¹ and R² each independently comprises optionally substituted alkyl.

4. The compound of claim 1, wherein the compound is an ionized form of general formula (1).

5. The compound of claim 2, wherein the compound is an ionized form of general formula (1A), where —C(═O)OR¹³ and/or —C(═O)OR¹⁴ has been ionized to become a negatively charged group.

6. The compound of claim 1, wherein the cationic therapeutic and/or cationic prophylactic agent comprises an antimicrobial agent.

7. The compound of claim 1, wherein the cationic therapeutic and/or cationic prophylactic agent comprises polymyxin.

8. (canceled)

9. (canceled)

10. A nanoparticle composition for delivery of a cationic therapeutic and/or cationic prophylactic agent, the nanoparticle composition comprising:
a compound as claimed in claim 1; and
a cationic therapeutic and/or cationic prophylactic agent that is encapsulated in said compound as claimed in claim 1.

11. The nanoparticle composition of claim 10, wherein the charge ratio of the cationic therapeutic and/or cationic prophylactic agent that is encapsulated in the compound as claimed in claim 1 to said compound is from 1:1 to 10:1.

12. The nanoparticle composition of claim 10, wherein the composition further comprises:
(a) helper lipid;
(b) sterol; and
(c) polyethylene glycol (PEG)-modified lipid.

13. The nanoparticle composition of claim 12, wherein the cationic therapeutic and/or cationic prophylactic agent, helper lipid, sterol, compound represented by general formula (1), and PEG-modified lipid are mixed at a molar ratio of 10-120:3-30:10-130:2-30:0.5-10.

14. The nanoparticle composition of claim 12, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin and combinations thereof.

15. The nanoparticle composition of claim 12, wherein the sterol is selected from cholesterol, fecosterol, sitosterol, ergosterol, campesterol, stigmasterol, brassicasterol, avenasterol and combinations thereof.

16. The nanoparticle composition of claim 12, wherein the PEG-modified lipid is selected from PEG-modified phosphatidylethanolamines, PEG-modified phosphatidic acids, PEG-modified ceramides, PEG-modified dialkylamines, PEG-modified diacylglycerols, PEG-modified dialkylglycerols, or the like or combinations thereof. Examples of PEG-modified/PEGylated lipid include, but is not limited to, 2-[(polyethylene glycol)-2000]N,N-ditetradecylacetamide (ALC-0159), R-3-[(ω-methoxy-poly(ethylene glycol)2000) carbamoyl]-1,2-dimyristyloxlpropyl-3-amine (PEG-c-DOMG), 3-N-[(ω-methoxypoly(ethyleneglycol)2000) carbamoyl]-1,2-dimyristyloxy-propylamine (PEG-S-DMG), PEG-DMPE (1,2-dimyristoyl-sn-glycero-3-phosphoethanolamine-N-[(polyethylene glycol)-methoxy] (sodium salt)), PEG-DPPC, PEG-DSPE lipid and combinations thereof.

17. The nanoparticle composition of claim 10, wherein the nanoparticle composition comprises nanoparticles having an average particle size of from 20 nm to 200 nm.

18. The nanoparticle composition of claim 10, wherein the nanoparticle composition comprises nanoparticles having a zeta potential of from −20 mV to +20 mV.

19. The nanoparticle composition of claim 10, wherein the nanoparticle composition comprises nanoparticles having a minimum bactericidal concentration (MBC) to minimum inhibitory concentration (MIC) ratio that is no more than 6.

20. (canceled)

21. (canceled)

22. (canceled)

23. A method of treating or preventing a disease, disorder or condition in a subject in need thereof, the method comprising administering a therapeutically effective amount of the nanoparticle composition as claimed in claim 10 to the subject, optionally wherein the disease, disorder or condition comprises bacterial infection.

24. (canceled)

25. The method of claim 23, wherein the disease, disorder or condition is mediated by Gram-negative bacteria.

26. The method of claim 25, wherein the Gram-negative bacteria is selected from the group consisting of Escherichia coli, Pseudomonas aeruginosa, and combinations thereof.
